(19) United States
(12) Patent Application Publication
(10) Pub. No.: US 2020/0271647 A1
(43) Pub. Date: Aug. 27, 2020

(54) BIOMARKERS FOR PREDICTING PROGNOSIS OF KIDNEY DISEASE

(71) Applicants: SEOUL NATIONAL UNIVERSITY HOSPITAL, Seoul (KR); Helixmith Co., Ltd., Seoul (KR); SEOUL NATIONAL UNIVERSITY R&DB FOUNDATION, Seoul (KR)

(72) Inventors: Jung Pyo LEE, Seoul (KR); Yong Chul KIM, Seoul (KR); Seung Hee YANG, Seoul (KR); Jae Gyun JEONG, Seoul (KR)

(21) Appl. No.: 16/721,073

(22) Filed: Dec. 19, 2019

(30) Foreign Application Priority Data
Dec. 19, 2018 (KR) 10-2018-0164858

(51) Int. Cl. G01N 33/566; G01N 33/68; G01N 33/70; C07K 16/42

(52) U.S. Cl. CPC G01N 33/566; G01N 33/6854; G01N 33/70; A61K 2039/505; G01N 2800/347; C07K 2317/565; C07K 16/42

(57) ABSTRACT

A kit for diagnosing a renal disease or predicting prognosis of the renal disease from urine includes a binding molecule specifically bound to cMet protein contained in the urine. When a level of cMet protein in urine is higher than a normal group, this condition is diagnosed as the renal disease or predicted to have poor prognosis thereof. A screening method of a substance for treatment of renal disease includes first-measuring a level of cMet protein in urine of an object, injecting a sample to be analyzed into the object, and second-measuring the level of cMet protein in urine of the object. When the level of cMet protein in the second-measuring is lower than the level of cMet protein in the first-measuring, the sample to be analyzed is the substance for treatment of renal disease.

### CROSS-REFERENCE TO RELATED APPLICATION

This application claims the benefit of Korean Patent Application No. 10-2018-0164858 filed on Dec. 19, 2018 in the Korean Intellectual Property Office, the disclosure of which is incorporated herein by reference in its entirety.

### BACKGROUND

### Technical Field

The present invention relates to a biomarker for predicting prognosis of renal disease.

### Background Art

Excessive protein such as albumin found in urine is a sign of indicating a renal disease. Diabetic renal disease is one of such diseases. Since excessive albumin is first detected, the renal disease is irreversible and may progress to a stage with reduced therapeutic effects. Therefore, it is important to develop a faster and more accurate test method than known radioimmunoassay, so as to early diagnose a disease and thus to prevent or begin treatment of the disease. Proteinuria such as urine containing albumin may be a disease marker to indicate diverse renal diseases including, for example, diabetic nephropathy, bacterial diabetic nephropathy and viral diabetic nephropathy, IgA nephropathy, Henoch-Schonlein Purpura, membranoproliferative glomerulonephritis, membranous nephropathy, Sjogren's syndrome, nephritic syndrome, acute kidney injury, acute tubulo-interstitial nephritis, acute pyelonephritis, preeclampsia, kidney transplant rejection, leprosy, reflux nephropathy, nephrolithiasis, renal cystic nephropathy, polycystic kidney disease, autosomal dominant polycystic kidney disease, autosomal recessive polycystic kidney disease, tuberous sclerosis, von Hippel-Lindau disease, familial thin-glomerular basement membrane disease, collagen III glomerulopathy, renal fibrosis glomerulopathy, Alport's syndrome, Fabry's disease, Nail-Patella syndrome, congenital urinary abnormality, multiple myeloma, amyloidosis, monoclonal nephropathy, Familial Mediterranean Fever (FMF), HIV infection (AIDS), systemic vasculitis, polyarteritis nodosa, Wegener's granulomatosis, aortitis, necrotic and crescentic glomerulonephritis, polymyositis, dermatitis, pancreatitis, rheumatoid arthritis, systemic erythematous lupus, gout, blastocyte disease, thrombosis, purpuric oral disease, hemolytic uremic syndrome, acute cortical necrosis, renal thromboembolism, renal cystadenoma, melanoma, lymphangioma, multiple myeloma, myocardial infarction, cardiac arrest, peripheral vascular disease, hypertension, coronary arterial heart disease, non-atherosclerotic cardiovascular disorder, atherosclerotic cardiovascular disorder, psoriasis, systemic sclerosis, COPD, occlusive sleep apnea syndrome, high altitude hypoxia, acromegaly, diabetes and insipidus. The renal disease may occur due to microbiosis, allergy, birth defects, stone, antibiotics, immunosuppressant, anticancer drugs, non-steroidal anti-inflammatory drugs, analgesics, heavy metals, tumor, chemicals or the like.

Among methods for measuring albumin, HABA or BCG method as a colorimetric method, salting out as a precipitation method, or the like is known in the art. The colorimetric method generally measures albumin using properties of albumin to couple to HABA or BCG pigment. However, the HABA method entails disadvantages of inaccuracy, poor sensitivity, instable color fixation by temperature in a case of hemolysis or cloudy serum, and the BCG method also entails disadvantages of inaccuracy, poor sensitivity, and temperature- and pH-sensitivity in a case of hemolysis or cloudy serum. Further, the salting out method entails disadvantages wherein other proteins and impurities may be easily precipitated along with albumin, hence not enabling keen discrimination, and needs a high protein concentration and a desalting operation.

Meanwhile, LI Xing et al., “Expression of cMet Stimulated by High Glucose in Human Renal Tubular Epithelial Cells and Its Implication,” Journal of Huazhong University of Science and Technology, 27 (2): 161-163, 2007 discloses that cMet plays an important role in development of diabetic nephropathy. Further, Youhua Liu, et al., “In vivo and in vitro evidence for increased expression of HGF receptor in kidney of diabetic rat,” American Journal of Physiology: Renal Physiology, 271(6): F1202-F1210, 1996, discloses that cMet expression is increased in the kidney of a diabetic rat. Correlation between cMet and the diabetic nephropathy has already been studied, however, such conventional studies relate to cMet in kidney tissues, which is a biomarker with difficulties in use thereof unless tissue inspection is implemented.

Further, renal tissue inspection is usually used for accurate diagnosis of glomerulonephritis, or the like, however, may entail a risk of complications such as hematoma around the kidney, visual hematuria, infection, arteriovenous fistula, etc., due to tissue examination. In order to treat the complications, surgical treatment such as catheter insertion is necessary, and therefore, new assay capable of simply inspecting kidney functions is required.

### SUMMARY

An object of the present invention is to provide a method for diagnosing a renal disease or predicting prognosis thereof without typical kidney tissue inspection.

1. A kit for diagnosing renal disease or predicting prognosis of the renal disease from urine, including: a binding molecule specifically bound to cMet protein contained in the urine,

wherein, when a level of cMet protein in urine (“level of cMet protein in urine”) is higher than a normal group, this condition is diagnosed as the renal disease or predicted to have poor prognosis of the renal disease by the kit.

2. The kit according to item 1, wherein the binding molecule is an anti-cMet antibody.

3. The kit according to item 1, wherein the renal disease is at least one selected from the group consisting of acute kidney injury, end-stage kidney disease (ESKD), systemic erythematous lupus, diabetic nephropathy (DN), IgA nephropathy (IgAN), HIV-relevant nephritis, non-diabetic chronic nephropathy, focal segmental glomerulosclerosis (FSGS), minimal change disease (MCD) and xanthine oxidase deficiency disease.

4. The kit according to item 1, further including a binding molecule specifically bound to creatinine.

5. A composition for diagnosing renal disease or predicting prognosis of the renal disease from urine, including: a binding molecule specifically bound to cMet protein contained in the urine, wherein, when a level of cMet protein is higher than a normal group, this condition is diagnosed as the renal disease or predicted to have poor prognosis of the renal disease by the composition.

6. A method for diagnosing renal disease or predicting prognosis of the renal disease from urine, including: measuring a level of cMet protein contained in a urine sample obtained from an object, wherein, when the level of cMet protein is higher than a normal group, this condition is diagnosed as the renal disease or predicted to have poor prognosis of the renal disease by the method.

7. The method according to item 6, wherein the level of cMet protein is measured using a binding molecule specifically bound to cMet.

8. The method according to item 6, wherein the binding molecule is an anti-cMet antibody.

9. The method according to item 6, wherein the normal group is an object without any renal disease.

10. The method according to item 6, wherein the renal disease is at least one selected from the group consisting of acute kidney injury, end-stage kidney disease (ESKD), systemic erythematous lupus, diabetic nephropathy (DN), IgA nephropathy (IgAN), HIV-relevant nephritis, non-diabetic chronic nephropathy, focal segmental glomerulosclerosis (FSGS), minimal change disease (MCD) and xanthine oxidase deficiency disease.

11. The method according to item 6, further including measuring a urine creatinine concentration, a total urinary protein concentration, or the urine creatinine concentration and the total urinary protein concentration.

12. A screening method of a substance for treatment of renal disease, including: a first measurement step of measuring a level of cMet protein in urine of an object; a step of injecting a sample to be analyzed into the object; and a second measurement step of measuring the level of cMet protein in urine of the object, wherein, when the level of cMet protein in the second measurement stage is lower than the level of cMet protein in the first measurement stage, the sample to be analyzed is the substance for treatment of renal disease and the object is any mammal other than a human.

13. A method for treatment of renal disease, including: measuring a level of cMet protein contained in a urine sample obtained from an object; and administering an anti-cMet antibody to the object with the level of cMet protein in urine higher than that of a normal group.

14. The method according to item 13, wherein the level of cMet protein is measured using a binding molecule specifically bound to cMet.

15. The method according to item 14, wherein the binding molecule is an anti-cMet antibody.

16. The method according to item 13, wherein the normal group is an object without any renal disease.

17. The method according to item 13, wherein the renal disease is at least one selected from the group consisting of acute kidney injury, end-stage kidney disease (ESKD), systemic erythematous lupus, diabetic nephropathy (DN), IgA nephropathy (IgAN), HIV-relevant nephritis, non-diabetic chronic nephropathy, focal segmental glomerulosclerosis (FSGS), minimal change disease (MCD) and xanthine oxidase deficiency disease.

18. The method according to item 13, further including measuring a urine creatinine concentration, a total urinary protein concentration, or the urine creatinine concentration and the total urinary protein concentration.

19. The method according to item 13, wherein the anti-cMet antibody includes: a heavy chain complementarity determining region having an amino acid sequence represented by SEQ ID NO: 1 (HCDR1); a heavy chain complementarity determining region having an amino acid sequence represented by SEQ ID NO: 2 (HCDR2); a heavy chain complementarity determining region having an amino acid sequence represented by SEQ ID NO: 3 (HCDR3); a light chain complementarity determining region having an amino acid sequence represented by SEQ ID NO: 4 (LCDR1); a light chain complementarity determining region having an amino acid sequence represented by SEQ ID NO: 5 (LCDR2); and a light chain complementarity determining region having an amino acid sequence represented by SEQ ID NO: 6 (LCDR3).

20. The method according to item 13, wherein the anti-cMet antibody includes: a heavy chain variable region having an amino acid sequence represented by SEQ ID NO: 7, 9, 10 or 11; and a light chain variable region having an amino acid sequence represented by SEQ ID NO: 8 or 12.

According to the present invention, a composition, a kit and a method for diagnosing a renal disease or predicting prognosis thereof from human urine have advantages of non-invasive and inexpensive.

The method for diagnosing a renal disease predicting prognosis thereof from human urine according to the present invention can reliably diagnose the renal disease or predict prognosis thereof by comparing variation over time even without clinical observations.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A and 1B are graphs illustrating correlation between cMet level and kidney function. *P<0.05, **P<0.01, and ***P<0.001.

FIGS. 2A and 2B are graphs illustrating classification of cMet levels in urine (‘urine cMet levels’) by kidney function (A), and proteinuria in the urine (B). **P<0.01, and ***P<0.001.

FIGS. 3A to 3C are graphs illustrating survival rates in Kaplan-Meier patients with respect to ESRD (A), all-cause mortality (B), and combined results (C).

FIG. 4A is images illustrating results of immunofluorescence assay for endothelial cells of glomerulus, podocytes, mesangial cells and proximal tubular epithelia cells (PTEC) (DAPI: blue, cMet: green, and CD31, nephrin, desmin and AQP1: red); and FIG. 4B is images illustrating an increase in phosphorylated cMet level in glomerulus of a patient suffering from diabetic nephropathy, as compared to experimental object in a normal control group (DAPI: blue, and phosphorylated cMet: red).

FIG. 5 is Kaplan-Meier survival curves illustrating survival periods of both patient groups having urine-cMet concentration of more than a median value (blue) and less than the same (green) among IgA nephritis patients.

FIG. 6 is a graph illustrating measured results of cMet protein concentration in urine with respect to different groups including: sham; sham with anti-cMet antibody administration; UIRI; and UIRI with anti-cMet antibody administration.

FIG. 7 is a photograph and a graph illustrating the kidney (left) extracted from each of different groups including: sham; sham with anti-cMet antibody administration; UIRI; and UIRI with anti-cMet antibody administration, and the weight of the left kidney to body weight.

FIG. 8 is images illustrating MT staining results of the kidney tissues shown in FIG. 7.

FIG. 9 images illustrating Sirius staining results of the kidney tissues shown in FIG. 7.

FIG. 10 is graphs numerically expressing areas of fibrinolyzed portions in FIGS. 8 and 9.

FIG. 11 is graphs illustrating verified results of the increase in expression of Fibronectin, Collagen1 and α-SMA in the kidney of each of different groups including: sham; sham with anti-cMet antibody administration; UIRI; and UIRI with anti-cMet antibody administration, respectively, through RT-PCR.

FIG. 12 a diagram illustrating verified results of Fibronectin, Collagen1 and α-SMA protein levels in the kidney of each of different groups including: sham; sham with anti-cMet antibody administration; UIRI; and UIRI with anti-cMet antibody administration, respectively, through Western blotting.

FIG. 13 is graphs of quantification of the protein levels from the images in FIG. 12.

FIG. 14 is graphs illustrating verified results of expression levels of IL-1β, TNF-α and Mcp-1 in the kidney of each of different groups including: sham; sham with anti-cMet antibody administration; UIRI; and UIRI with anti-cMet antibody administration, respectively, through RT-PCR.

FIG. 15 is a graph illustrating migration effects to HUVEC, in order to compare biopotencies between selected candidate antibodies in the present invention.

### DETAILED DESCRIPTION

Hereinafter, the present invention will be more sufficiently described below with reference to the accompanying drawings. Further, preferred embodiments will be illustrated as a part of the present invention rather than a whole thereof. In fact, the present invention can be implemented in diverse forms and should not be construed to be limited to the proposed embodiments. The singular form used in the specification and the appended claims may also include plural subjects unless otherwise clearly specified.

In one embodiment of the present invention, there is provided a composition and/or a kit for diagnosis of a renal disease from urine, which includes a binding molecule specifically bound to cMet protein contained in the urine. According to one embodiment of the present invention, the binding molecule may be an antibody or a variant thereof.

Further, in one embodiment of the present invention, there is provided a method for predicting prognosis of a renal disease, which includes contacting a composition including the binding molecule specifically bound to cMet protein contained in a sample. According to one embodiment of the present invention, the sample is preferably urine.

According to one embodiment of the present invention, cMet concentration in the urine of an experimental object (“an object”) may be measured once, and then compared with cMet concentration in the urine of a normal person. In this case, when the cMet level in urine (‘urine cMet level’) of the object is higher than that of the normal person, the object may be diagnosed to have the renal disease or predicted to show poor prognosis of the renal disease. The normal person may include, for example, persons without cMet relevant diseases, persons who are diagnosed with no renal disease, or persons without any type of disease, and so on. The urine cMet concentration of the normal person may be previously investigated or, otherwise, an average of urine cMet concentrations of at least one, at least 2, at least 5, at least 10 or at least 20 normal persons.

In another embodiment of the present invention, the level of cMet protein contained in the urine is measured at least twice, at least 3 times, at least 4 times or at least 5 times at a predetermined time interval, and when the level of cMet protein is increased or showing a tendency of increasing over time, the object may be diagnosed to have the renal disease or predicted to show poor prognosis of the renal disease. According to one embodiment of the present invention, the predetermined time interval is a time interval to be considered clinically meaningful and may be, for example, 3 months or more.

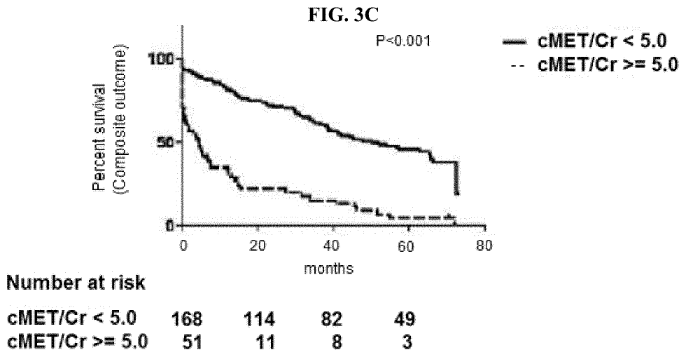

cMet is expressed in the glomerulus of a healthy object (FIG. 4A). Meanwhile, in the present invention, it was confirmed that a concentration of soluble cMet or cMet/Cr level detected in the urine is increased as the prognosis of a patient suffering from the renal disease is worse. Since cMet expression is increased in human glomerular epithelial cells (GEC), soluble sMet observed in the urine is deemed to be derived from the kidney (Li, J. et al. Blockade of endothelial-mesenchymal transition by a Smad3 inhibitor delays the early development of streptozotocin-induced diabetic nephropathy. Diabetes 59, 2612-2624 (2010)), (Wajih, N., Walter, J. & Sane, D. C. Vascular origin of a soluble truncated form of the hepatocyte growth factor receptor (cMet). CircRes 90, 46-52 (2002).).

According to the present invention, a urine cMet/Cr level at the diagnosis of diabetic nephropathy is significantly relevant to urine protein-to-creatinine ratio (UPCR), and shows reverse correlation with estimated GFR (eGFR). However, cMet/Cr was generally increased in patients in chronic kidney disease (CKD) stage V. This result means that cMet filtration is increased when kidney functions become worse.

As compared to common diagnosis markers such as serum creatinine and UPCR, cMet may exhibit utility as a biomarker alone in ROC assay. In order to identify end-stage renal disease (ESRD) expectation, additional expectation of urine cMet/Cr has been assessed during diagnosis of the diabetic nephropathy. The ESRD patients exhibited a relatively high level of soluble urine cMet/Cr. The urine can be collected by non-invasive methods, and is the useful origin of protein for biomarker analysis. Therefore, using a urine sample to identify novel biomarkers is clinically significant. However, the urine sample is very diverse in volume, protein concentration, total protein content and pH, and may be perishable during storage. Considering such problems, cMet ELISA kit was assessed with some indicators such as LLOQ, dilution linearity and parallelism.

In one embodiment of the present invention, a model for prediction or diagnosis may be provided on the basis of a level of cMet protein contained in the urine. This model may be a form of, for example, software code, computer-readable format or paper instruction for assessment of relative expression of biomarker.

As used herein, the term “kidney (or renal) disease” refers to any disease causing damage to the kidney and, for example, may include, without limitation thereof, at least one selected from the group consisting of acute kidney injury, end-stage kidney disease (ESKD), systemic erythematous lupus, diabetic nephropathy (DN) or diabetes mellitus nephropathy (DMN), IgA nephropathy (IgAN), HIV-relevant nephritis, non-diabetic chronic nephropathy, focal segmental glomerulosclerosis (FSGS), minimal change disease (MCD) and xanthine oxidase deficiency disease.

Acute kidney injury refers to a disease with rapid deterioration in kidney functions, which may include from tiny change in kidney function to kidney failure in serious stage requiring novel alternative therapy. Depending upon changes in serum creatinine level and urine amount, this disease may be divided into three stages including “Risk,” “Injury” and “Failure”. Based on final results of kidney injury, “Loss” and “End-Stage Kidney Disease” categories may also be included. Each of these categories may be classified as follows:

Risk: 1.5-fold increase in creatinine level or reduction of more than 25% in glomerular filtration rate (GFR), and urine amount of less than 0.5 mL/kg/h×6 hrs.;

Injury: 2-fold increase in creatinine level or reduction of more than 50% in GFR, and urine amount of less than 0.5 mL/kg/h×12 hrs.;

Failure: 3-fold increase in creatinine level or reduction of more than 75% in GFR, and urine amount of less than 0.3 mL/kg/h×24 hrs. or no excretion of urine (anuria) for 12 hours;

Loss: continuous acute renal failure complete loss of kidney function for more than 4 months;

ESKD: end-stage renal disease for more than 3 months (Bellomo R, et al., Acute Dialysis Quality Initiative workgroup. Acute renal failure-definition, outcome measures, animal models, fluid therapy and information technology needs: the Second International Consensus Conference of the Acute Dialysis Quality Initiative (ADQI) Group. Crit Care 2004; 8: R204-R212.).

If the object has a problem in kidney function or kidney structure for 3 months or more, the object may be diagnosed to have chronic kidney disease (CKD). CKD is principally divided into stage I to stage V based on whether the kidney function is activated well or not. In the initial stage (stage I), the kidney can still filter body wastes from the blood, whereas filtering function is not performed in the final stage (stage V) and the kidney almost stops its action. The stages may be classified into as follows according to GFR levels:

Stage I: GFR≥90;

Stage II: 60≤GFR<90;

Stage IIIa: 45≤GFR<60;

Stage IIIb: 30≤GFR<45;

Stage IV: 15≤GFR<30;

Stage V: GFR<15.

GFR indicates an amount of body fluid filtered per unit time. The estimated GFR (eGFR) may be calculated with blood creatinine concentration, age, gender and race as variable parameters.

Creatinine (Cr) is a by-product from muscle metabolism, which is removed from blood and excreted through urine by the healthy kidney. A predetermined amount of Cr per day is usually discharged through urine. In order to measure proteinuria in the object, urine assay for 24 hours will be most accurate, however, due to actual difficulties, a ratio of Cr to specific proteins contained in the urine is calculated and used for determining the kidney function irrespective of time. Urine Cr concentration may be measured, for example, by a Jaffe method, liquid chromatography, mass spectrometry or ELISA, but it is not limited thereto. Instead, this may be determined by any method well known in the art to which the present invention pertains.

cMet (NP_000236, NP_001120972, NP_001311330 or NP_001311331) is known as tyrosine-protein kinase Met or a hepatocyte growth factor receptor (HGFR), which is a protein encoded by MET gene in the human. This has tyrosine kinase activity and plays essential roles in development of embryo bud, formation of organs and injury treatment. The hepatocyte growth factor receptor and truncated homogenous proteins thereof, that is, NK1 and NK2 are ligands of MET receptor. Both HGF and cMet are known to be expressed in different cells. It is known that a process of transmitting a HGF-cMet signal is associated with a variety types of cancers, neurodegenerative diseases, peripheral vascular tract diseases, etc. Accordingly, diverse types of formulations capable of activating or inhibiting transmission of the HGF-MET signal have been developed.

Cox proportional hazards model is a statistic assay model to prepare a pattern for prediction of time-death data. Observations should be independent and need “proportional risk hypothesis” wherein a risk rate is constant indifferent to time. Cox model is a multivariate assay method broadly used when comparing survival rates between groups while controlling different confounding variables or, otherwise, in order to investigate simultaneous effects of different variables on survival time.

Kaplan-Meier survival curve adopts a method of estimating cumulative survival rate from accumulation of sectional survival rates at every death time. Kaplan-Meier survival assay is also called a product-limit method. In the order of observation period, the sectional survival rate (P(t)) is calculated in a fraction of survived ones by percentage among the total number of observed subjects. The cumulative survival rate (S(t)) may be estimated by multiplying the sectional survival rates in sequential order.

As used herein, the term “diagnosis” refers to determining sensitivity of a single object to specific disease or disorder, determining whether the object has the specific disease or disorder now or not, determining prognosis of the object with the specific disease or disorder, and/or therametrics.

As used herein, the term “kit for diagnosis of renal disease” refers to a kit including a composition for diagnosing a renal disease. The kit may further include instruction to explain a method of using the kit.

As used herein, the term “biomarker,” “protein marker,” “diagnosis marker,” “marker for diagnosis,” “diagnostic marker,” etc. may be interchangeably used and may refer to a bio-labeling material present in a living body, and means a substance that can exhibit or expect a response to diagnosis, prognosis or treatment of a disease. Further, this may also include organic bio-molecules such as polypeptide or nucleic acid (e.g., DNA, mRNA, etc.), lipids, glycolipids, glycoproteins, saccharides (monosaccharides, disaccharides, oligosaccharides, etc.) exhibiting a tendency of increasing in cells derived from patients with renal disease in a high-risk group, as compared to cells derived from patients with renal disease in a low-risk group. In one embodiment of the present invention, the marker for diagnosis of renal disease is cMet protein found in the urine that exhibits an increased expression level in the cells derived from patients with renal disease in the high-risk group.

As used herein, the term “measurement” refers to a method that includes detecting the presence or absence of a marker in a sample, measuring an amount of the marker in the sample, and/or identifying a specific type of biomarker. The measurement may be performed by a method known in the art and a method further described in the present invention, in particular, may include, without limitation thereof, SELDI and immunoassay. In order to detect and determine at least one among markers described in the present disclosure, any suitable method may be used. Such methods may include, without limitation thereof, ELISA, western blotting, mass spectrometry (e.g., laser desorption/ionization mass spectrometry), fluorescence (e.g., sandwich immunoassay), surface plasmon resonance, elliptical measurement and atomic microscopy.

Those skilled in the art will understand that a wide range of various analytic techniques could be utilized to determine a level of biomarker, as described in the specification and the appended claims. A different type binding reagent useable by those skilled in the art may be used to determine a level of the indicated matter to be analyzed (“analyte”) in the sample. For example, diverse binders or binding reagents suitable for evaluating a level of a given analyte may be easily identified. Generally, a proper binder may be specifically bound to the analyte, in other words, may react with the analyte in a detectable level. On the other hand, this agent does not react with other analytes or irrelevant analytes in a detectable level (or showing reaction with limited cross-reactivity). It is considered that the proper binder may include polyclonal and monoclonal antibodies, aptamer, RNA molecules or the like. Immunofluorescence staining, mass spectrometry, nuclear magnetic resonance and spectroscopic methods including photo-spectroscopic analysis, etc. may also be used to determine the level of analyte. According to binders utilized herein, a sample may be processed, for example, by dilution, purification, denaturation, degradation, fragmentation, etc., and then, analyzed as known to those skilled in the art.

It is considered that the identified biomarker may include a binding region with respect to various epitopes and/or other type binders for immunoassay. Accordingly, it is considered that a peptide fragment or other epitope of the identified biomarker, iso-type of specific protein, and even a compound present on upstream or downstream of a biological route or modified after translation may replace the identified analyte or the biomarker as long as stoichiometric theory relevant to and relative to the above matters is desirably regarded. Those skilled in the art will recognize that an alternative antibody and binder may be used in order to determine the level of any random and specific analyte as long as different specific properties and binding affinity of an alternative antibody and binder are considered in the analysis.

In order to measure or determine the expression level of an analyte or biomarker used in the method and the test kit according to the present invention, a variety of algorithms may be used. Such an algorithm as described above is generally considered to determine an analyte level beyond simple measurement of cut-off values. Therefore, a result of the algorithm may be classified as multi-index inspection by the U.S. Food and Drug Administration (U.S. FDA). Specific types of algorithms may include knowledge discovery engine (KDE™ regression analysis, discriminant analysis, classification tree analysis, random forest, PROTEOMEQUEST® support vector machine, One R, kNN and Heuristics Naïve Bayes analysis), neural network and modification thereof.

As used herein, the term “antibody” or “binding protein” refers to specific protein molecules indicated with respect to antigenic sites. Based on the purposes of the present invention, the antibody may refer to an antibody specifically bound to a marker protein, and include all of polyclonal antibodies, monoclonal antibodies and recombinant antibodies. Not only a whole antibody form but also functional fragments of an antibody molecule may also be included. The whole antibody may have a structure consisting of two full-length light chains and two full-length heavy chains, wherein each light chain is connected to a heavy chain though a disulfide bond. The functional fragment of an antibody molecule means a fragment with antigen-combining function and may include Fab, F(ab′), F(ab′)₂, Fv, etc. Among those antibody fragments, Fab has a structure including variable regions in the light chain and heavy chain, a constant region in the light chain, and a first constant region (CH1) in the heavy chain, wherein one antigen-combining site is included. Fab′ has a hinge region including at least one cysteine residue at C terminal of CH1 domain in the heavy chain, and in this aspect, thus is different from Fab. F(ab′)2 antibody is produced as the cysteine residue at the hinge region of Fab′ forms a disulfide bond. Fv is a minimum antibody fragment having a heavy chain variable site and a light chain variable site only, wherein recombination technique to produce Fv fragments was disclosed in WO Patent Publication Nos. WO 88/10649, WO 88/106630, WO 88/07085, WO 88/07086 and WO 88/09344. Double-chain Fv (dsFv) is characterized in that a heavy chain variable site and a light chain variable site are connected through the disulfide bond, while single-chain Fv (scFv) includes a heavy chain variable region and a light chain variable region connected by a covalent bond through a peptide linker. Such an antibody fragment may be obtained using protein hydrolase (“protease”), preferably, produced according to genetic recombination technology. In the present invention, an antibody is preferably in a form of Fab or a whole antibody. In the present invention, the antibody may be conjugated with microparticles. In addition, the microparticles may be colored latex or colloidal gold particles. In the present invention, the antibody may be any antibody wherein an expression level of protein encoded by known mRNA gene with respect to the marker described above can be measured. For example, the kit may include an immunoassay kit, but it is not limited thereto. According to one embodiment, the kit is a Luminex assay kit, a protein microarray kit or ELISA kit.

The Luminex assay kit, protein microarray kit and ELISA kit may include polyclonal antibody and monoclonal antibody to the protein marker of the present invention, and secondary antibody to the polyclonal antibody and monoclonal antibody, which are combined with a labeling agent.

Examples of the kit in the present invention may include immune-chromatography strip kit, Luminex assay kit, protein microarray kit, ELISA kit, immune dot kit, etc. but it is not limited thereto.

In addition, the kit may further include essential elements necessary for ELISA. The ELISA kit may include an antibody specific to a marker protein. This antibody is one having high specificity and affinity to a marker protein while substantially not having cross-reactivity with other proteins, and may include a monoclonal antibody, a polyclonal antibody or a recombinant antibody. Further, the ELISA kit may include an antibody specific to a control protein. In addition, the ELISA kit may include a reagent capable of detecting combined antibodies, for example, labeled secondary antibodies, chromophores, enzymes and substrates thereof, other materials possibly combined with the antibody, etc.

Further, the kit may include essential elements necessary for protein microarray in order to simultaneously analyze complex markers. The microarray kit may include an antibody specific to a marker protein combined with a solid phase. The antibody is one having high specificity and affinity to a marker protein while substantially not having cross-reactivity to other proteins, and may include a monoclonal antibody, a polyclonal antibody or a recombinant antibody. Further, the protein microarray kit may include an antibody specific to a control protein. In addition, the protein microarray kit may include a reagent for detecting combined antibodies, for example, labeled secondary antibody, chromophores, enzymes and substrates thereof, other materials possibly combined with the antibody, etc. A sample assay method using the protein microarray may include isolating protein from the sample, hybridizing the isolated protein with a protein chip to form an antigen-antibody complex, reading the same to identify the presence of protein or an expression level thereof, thereby providing information required for diagnosis of renal disease.

Measurement of protein expression level may be implemented using an ELISA method. The ELISA method may include various ELISA methods such as: direct ELISA using labeled antibody that recognizes an antigen adhered to a solid support; indirect ELISA using the labeled antibody that recognizes a capture antibody in an antibody complex recognizing an antigen adhered to a solid support; direct sandwich ELISA using another labeled antibody that recognizes the antigen in an antibody-antigen complex adhered to a solid support; indirect sandwich ELISA using labeled secondary antibody, wherein this secondary antibody reacts with another antibody recognizing the antigen in an antibody-antigen complex adhered to a solid support, and then, recognizes the above another antibody; or the like. More preferably, protein expression may be detected by sandwich ELISA, which includes: adhering an antibody to a solid support; reacting a sample with the antibody; adhering a labeled antibody, which recognizes the antigen in an antigen-antibody complex, to the above reacted antibody; and enzymatically coloring the above adhered antibody. Whether a renal disease occurs or not may be identified by determining an extent of forming a complex of a renal disease marker protein and an antibody.

Western blotting with at least one antibody to the renal disease marker may be used. After isolating all proteins from a sample, the proteins are subjected to electrophoresis to separate individual proteins according to a size thereof, followed by transferring the proteins toward a nitrocellulose membrane and reacting the same with an antibody. An amount of the produced antigen-antibody complex may be measured using the labeled antibody so as to determine an amount of the protein produced by gene expression, thereby identifying an occurrence of the renal disease. The above detection method may include investigating an amount of a marker protein in a sample obtained from a patient having the renal disease as well as an amount of the marker protein in a control group. A protein level may be indicated as absolute difference (e.g., μg/ml) or relative difference (e.g., a relative intensity of signal) in amounts of the marker protein.

A protein chip configured by aligning at least one antibody to the renal disease marker described above at a predetermined position on a board and fixing the same with a high density may be used. A sample assay method using the protein chip may include: isolating a protein from a sample; hybridizing the isolated protein with a protein chip to form an antigen-antibody complex; and reading the same to detect the presence of protein or an expression level thereof, thereby identifying an occurrence of any renal disease.

As used herein, the term “expression” refers to biological production of a product encoded by a coding sequence. In most cases, DNA sequence including the coding sequence may form a messenger-RNA (mRNA) through transcription. Then, the messenger RNA may be translated to form a polypeptide product having related biological activity. Further, expression may include an additional processing stage with respect to RNA transcript product (for example, splicing to remove intron), and/or translation-post-processing of the polypeptide product.

As used herein, a biological sample refers to tissues, cells, blood, serum, plasma, saliva, cerebrospinal fluid, or urine.

A gene expression level in the biological sample may be determined by measuring an amount of protein. A protein sequence used as a marker in the present invention may include polypeptides having sequence identity to the protein sequence.

With respect to amino acid sequence, the term “same or identical” indicates, as compared along with proper insertion or deletion, extent of identity between two nucleic acids or amino acid sequences.

Percentages of identity between two sequences may be represented by a logarithmic function in regard to the number of the same positions shared by the sequences (i.e., % identity=the number of the same positions/a total number of positions ×100), in consideration of: the number of gaps necessary to be introduced for optimum array of two sequences, and a length of each gap. Comparison of sequences and measurement of % identity between two sequences may be accomplished by a mathematical algorithm as described in the following non-limited examples. % identity between two amino acid sequences may also be determined according to an algorithm introduced in a document [E. Meyers and W. Miller (Comput. Appl. Biosci., 4:11-17 (1999))] integrated in ALIGN program (ver. 2.0) using PAM120 weighted value residue table, a gap length penalty of 12 and a gap penalty of 4. Further, % identity between two amino acid sequences may be determined according to an algorithm introduced in a document [Needleman and Wunsch (J. Mol. Biol. 48: 444-453 (1970))] integrated in GAP program of GCG software package using Blossum 62 matrix or PAM250 matrix, and a gap weighted value of 16, 14, 12, 10, 8, 6 or 4 and a length weighted value of 1, 2, 3, 4, 5 or 6.

For example, the polypeptide sequence of the present invention may be identical, that is, 100% identical to a reference sequence, otherwise, may include a change in the number of amino acids in a range of not more than specific integer or numerical number, as compared to the reference sequence, so that % identity becomes less than 100%. The change described above may be selected from the group consisting of at least one amino acid deletion, substitution including conservative and non-conservative substitution, or insertion, and may occur in any position such as an amino-terminal or a carboxy-terminal site in the reference polypeptide sequence, between the above terminals separately distributed among amino acids in the reference sequence, or within at least one continuous group in the reference sequence. The number of amino acid changes with respect to the given % identity may be determined by multiplying the total number of amino acids in the polypeptide sequence (divided by 100) with % in numerical value of each % identity, and then, subtracting the above multiplied value from the total number of amino acids in the polypeptide reference sequence or, otherwise, determined by the following equation.

nₐ≤xₐ−(xₐ·y)

(wherein nₐ is the number of amino acid changes, xₐ is the total number of amino acids in the polypeptide sequence, y is, for example, 0.70 for 70%, 0.80 for 80%, 0.85 for 85%, etc., and a random non-integer multiplication of xₐ and y is subjected to dropping fractions not higher than 0.5 to a nearest integer before subtraction of the same from xₐ.

Further, according to one embodiment of the present invention, there is provided a screening method of a substance for treatment of renal disease, which includes: a first measurement step of measuring a level of cMet protein in urine of an object; a step of injecting a sample to be analyzed into the object; and a second measurement step of measuring a level of cMet protein in urine of the object, wherein, when the level of cMet protein in the second measurement stage is identical to or less than the level of cMet protein in the first measurement stage, the sample to be analyzed is a substance for treatment of renal disease and the object is any mammal other than a human.

According to one embodiment of the present invention, there is provided a method for treatment of renal disease, which includes: measuring a level of cMet protein in urine of an object; comparing the result with a level of cMet protein in urine of a normal person; and then administering a therapeutic agent for renal disease to the object with a level of cMet protein in urine higher than that of the normal person.

According to one embodiment of the present invention, there is provided a method for treatment of renal disease by administering a renal disease therapeutic agent to a patient with a level of cMet protein in urine increasing over a predetermined time. In this regard, the level of cMet protein is measured at least twice at a predetermined time interval over the predetermined time, and those skilled in the art may control the number of measurements or the time interval as necessary. The patient showing an increase in the level of cMet protein in urine over the predetermined time is classified as a group expected to have poor prognosis of the renal disease, and the renal disease may be cured or the prognosis becomes better by administering the renal disease therapeutic agent to the corresponding patient.

According to one embodiment of the present invention, the renal disease therapeutic agent may include: a heavy chain complementarity determining region having an amino acid sequence represented by SEQ ID NO: 1 (HCDR1); a heavy chain complementarity determining region having an amino acid sequence represented by SEQ ID NO: 2 (HCDR2); a heavy chain complementarity determining region having an amino acid sequence represented by SEQ ID NO: 3 (HCDR3); a light chain complementarity determining region having an amino acid sequence represented by SEQ ID NO: 4 (LCDR1); a light chain complementarity determining region having an amino acid sequence represented by SEQ ID NO: 5 (LCDR2); and a light chain complementarity determining region having an amino acid sequence represented by SEQ ID NO: 6 (LCDR3). According to another embodiment of the present invention, the renal disease therapeutic agent may include: a heavy chain variable region having an amino acid sequence represented by SEQ ID NO: 7; and a light chain variable region having an amino acid sequence represented by SEQ ID NO: 8. Further, in one embodiment of the present invention, an anti-cMet antibody sequence according to the present invention may involve conservative amino acid substitution within a range that does not modify physiological activity.

The variable region refers to a portion of the antibody molecule showing variation a lot in sequence while functioning to specifically bind to an antigen, wherein CDR1, CDR2 and CDR3 are present in the variable region. A “complementarity determining region (CDR)” refers to a portion relevant to recognition of antigen, which is important because specificity of an antibody to an antigen is determined due to variation in sequence on the above portion. A “framework (FR) region” is present between CDRs in a proper orientation to support CDR rings, specifically, may include FR1, FR2, FR3 and FR4 present therein.

Conservational amino acid substitution means replacing an amino acid residue by another amino acid residue having a similar side chain. A family of amino acid residues having similar side chains is generally defined within the technical field to which the present invention pertains. This family may include amino acid having a basic side chain (e.g., lysine, arginine, histidine, etc.), amino acid having an acidic side chain (e.g., aspartic acid, glutamic acid, etc.), amino acid having a non-charged polar side chain (e.g., glycine, asparagine, glutamine, serine, threonine, tyrosine, cysteine, etc.), amino acid having a non-polar side chain (e.g., alanine, valine, leucine, isoleucine, proline, phenylalanine, methionine, tryptophane, etc.), amino acid having a beta-branched side chain (e.g., threonine, valine, isoleucine, etc.) and amino acid having an aromatic side chain (e.g., tyrosine, phenylalanine, tryptophan, histidine, etc.)

According to one embodiment of the present invention, the antibody may further include Fc region and may be a human antibody or humanized antibody. As used herein, the term “humanized antibody” totally refers to non-immunogenic antibodies and/or antibodies with decreased immunogenicity. The humanized antibody is an altered antibody with modified amino acid sequence, wherein the amino acid sequence of the antibody may be desirably re-constituted. Such possible changes may be innumerable and may range, for example, from changing one or some of amino acids to completely re-constituting a variable and/or constant region of the antibody. The variable region is typically altered to increase binding ability and affinity of an antigen, whereas, alteration in the constant region may be conducted in order to increase intracellular reaction such as fixation of complements, interaction with a membrane, functions of other effective agents, etc. The humanized antibody provided in the present invention may be combined with all types of constant regions by recombinant techniques. The heavy chain constant region may include gamma (γ), mu (μ), alpha (α), delta (5) and epsilon (s) types, in addition, subclass types such as gamma1 (γ1), gamma2 (γ2), gamma3 (γ3), gamma4 (γ4), alpha1 (α1) and alpha2 (α2). Further, the light chain constant region may include kappa (κ) and lambda (λ) types (Coleman et al., Fundamental immunology, 2nd Ed., 1989, 55-73).

With respect to IgG1-based anti-cMet antibody, that is, 1E4, disclosed in the existing patent publications Nos. WO2016/021864A1 and WO2017/135791A1, the present inventors have introduced CDR grafting technique to graft a complementarity determining region (CDR) in a framework region more stable than that of the existing antibody in order to improve structural stability while retaining characteristics of anti-cMet antibody.

“CDR grafting” has been developed to solve a problem of inducing immune response thus to lead neutralization when a mouse monoclonal antibody is used in a human patient, which is the most representative method for humanization of a non-human antibody. CDR grafting refers to transplanting CDR portion of an animal antibody to a framework of a human antibody.

The present inventors have applied the above CDR grafting method in order to select the optimum framework region. As a result, sequence homology between amino acid sequences that are present in the heavy chain variable region and light chain variable region of the human antibody, and amino acid sequences that are present in the heavy chain variable region (HV) and light chain variable region (Vk) of cMet antibody 1E4, was identified, a candidate sequence having high sequence homology was screened among the above sequences, and therefore, a variant of the anti-cMet antibody or the antigen combine fragment with improved stability was prepared according to the present invention.

According to one embodiment of the present invention, the anti-cMet antibody for treatment of the renal disease as described above may be provided in a form of a composition along with a pharmaceutically acceptable carrier. The pharmaceutically acceptable carrier included in the composition of the present invention is generally used in producing a formulation, and may include lactose, dextrose, sucrose, sorbitol, mannitol, starch, acacia rubber, calcium phosphate, alginate, gelatin, calcium silicate, microcrystalline cellulose, polyvinyl pyrrolidone, cellulose, water, syrup, methyl cellulose, methylhydroxy benzoate, propylhydroxy benzoate, talc, magnesium stearate and mineral oil, etc., but it is not limited thereto. Other than the above components, the pharmaceutical composition of the present invention may further include lubricants, wetting agents, sweeteners, flavors, emulsifiers, suspensions, preservatives and the like. Pharmaceutically acceptable carriers and formulations suitable in the art are described in detail in Remington's Pharmaceutical Sciences (19th ed., 1995).

As used herein, the expression “or a combination thereof” indicates all permutations and combinations of the previously listed items. For example, “A, B, C, or a combination thereof” may include A, B, C, AB, AC, BC, or ABC, and, if the listing order is important in specific situations, may include at least one among BA, CA, CB, CBA, BCA, ACB, BAC or CAB.

The following examples are proposed for illustrative purpose only and should be construed to limit the scope of the present invention under any circumstances.

### Example

Statistic Assay

Using 2.9 ng/ml of cMet cutoff or 4.9 ng/mg of cMet/Cr cutoff in urine, which were calculated from ROC curve, patients were divided into two groups. In order to determine influence of urine cMet or cMet/Cr level on ESRD and death, Kaplan-Meier survival curve classified by the urine cMet or cMet/Cr level was compared to log-rank tests.

At diagnosis of diabetic nephropathy, Cox proportional hazard models with regard to time-fixed urine cMet or cMet/Cr level, were used for multivariate survival analyses. Significant covariates found in univariate analysis and clinically significant covariates were added to the final multivariable-adjusted analysis, which in turn, was executed in a backward stepwise manner. The covariates used in separate models are as follows: model 1, age, gender, hypertension (high blood pressure), Hb, albumin, AST, ALT, uric acid, cholesterol, Ca and P; and model 2, age, gender, hypertension, Hb, albumin, AST, ALT, uric acid, cholesterol, Ca, P, GFR and PCR.

In order to examine increased prognostic values before and after adding a soluble urine cMet level to known variables such as serum creatinine and urine protein-to-creatinine ratio (UPCR), statistic significance of a difference between two types of areas under ROC curve (AUC) was estimated by the known method (DeLong, E. R., DeLong, et al., Biometrics 44, 837-845 (1988)). Additionally, a variation in estimation accuracy of the model was calculated as relative IDI and cfNRI according to the known method (Pencina, M. J., et al., Stat Med 27, 157-172, discussion 207-112 (2008).

All statistic assays were executed by SPSS version 22 (IBM, Chicago, Ill., United States) and R (version 3.2.5; The R Foundation for Statistical Computing, Vienna, Austria). If P value is less than 0.05, the results are demonstrated to be statistically significant.

### Example 1—Basic Characteristics and Soluble Urine cMet of Patient with Chronic Nephropathy

In the present invention, among 218 patients clinically diagnosed with diabetic nephropathy (DN), 20 patients received definite diagnosis with biopsy, except for patients scheduled to have a kidney transplant or patients aged of less than 18.

Age, gender, body mass index (BMI) and systolic and diastolic blood pressures at diagnosis, and, for example, demographic and clinical information such as accompanying disease, i.e., hypertension and diabetes have been acquired from medical records.

Experimental values including complete blood cell counts (CBCs), hemoglobin and aspartate aminotransferase (AST), alanine aminotransferase (ALT), albumin, urea, creatinine, calcium, phosphorus, cholesterol, glucose and HbA1c levels in serum were collected. Protein and creatinine levels in urine (“urine protein level and urine creatinine level”) were measured and a protein-to-creatinine ratio (UPCR) in urine was calculated.

The experimental value was measured by Modular D2400 analyzer having ISE900 module (Hitachi Ltd., Tokyo, Japan) and Cobas 8000 Modular analyzer (Roche Diagnostics, Basel, Switzerland). Further, eGFR was calculated with CKD-EPI creatinine formula.

The basic characteristics of participants are shown in Table 1 below. In this table, numerical values indicate a proportion (%) of patients in a specific category to a total patient, wherein 1 to 5 in first column denote stages I to V for CKD, respectively, and each numerical value are represented as a mean±standard deviation. Abbreviations are as follows. BMI: body mass index; SBP: systolic blood pressure; DBP: diastolic blood pressure; eGFR: estimated glomerular filtration rate (measured by CKD-EPI creatinine equation); PCR: protein-to-creatinine ratio.

TABLE 1

| | Total | 1 | 2 | 3 | 4 | 5 | P |
|---|---|---|---|---|---|---|---|
| Number of patients (n) | 218 | 15 | 19 | 58 | 63 | 63 | |
| Age (years) | 61.3 ± 13.9 | 41.6 ± 17.9 | 55.2 ± 16.0 | 64.0 ± 10.3 | 66.4 ± 11.6 | 60.5 ± 12.8 | <0.001 |
| Sex, male {n [%]} | 138 (63.3) | 10 (66.7) | 11 (57.9) | 41 (70.7) | 36 (57.1) | 40 (63.5) | |
| BMI (kg/cm²) | 24.0 ± 4.3 | 25.4 ± 4.8 | 25.0 ± 3.0 | 23.9 ± 3.9 | 24.8 ± 4.4 | 23.1 ± 4.7 | |
| SBP (mmHg) | 140.5 ± 28.4 | 133.9 ± 20.0 | 134.9 ± 22.2 | 143.2 ± 27.4 | 139.2 ± 27.9 | 142.2 ± 32.0 | |
| DBP (mmHg) | 75.1 ± 16.3 | 82.6 ± 14.4 | 79.0 ± 12.1 | 78.3 ± 14.9 | 73.1 ± 16.2 | 72.6 ± 18.0 | |
| cMet (ng/ml) | 3.8 ± 8.1 | 1.4 ± 1.6 | 1.1 ± 2.0 | 2.2 ± 5.9 | 2.2 ± 5.4 | 8.3 ± 11.8 | <0.001 |
| cMet (Creatinine (ng/mg) | 7.7 ± 21.1 | 1.3 ± 1.5 | 1.7 ± 3.7 | 4.6 ± 16.0 | 4.1 ± 12.6 | 17.7 ± 31.9 | <0.001 |
| Blood hemoglobin (d/gl) | 10.9 ± 2.2 | 12.9 ± 2.8 | 12.7 ± 2.9 | 11.8 ± 2.0 | 10.4 ± 1.4 | 9.7 ± 1.5 | <0.001 |
| White blood cell (/μl) | 7.3 ± 1.9 | 7.7 ± 1.8 | 7.3 ± 1.6 | 7.3 ± 1.4 | 7.4 ± 2.4 | 7.0 ± 2.0 | |

Serum

| | | | | | | | |
|---|---|---|---|---|---|---|---|
| Calcium (mg/dl( | 8.5 ± 0.8 | 8.9 ± 0.9 | 8.8 ± 0.6 | 8.7 ± 0.6 | 8.6 ± 0.6 | 8.0 ± 0.8 | <0.001 |
| Phosphorus (mg/dl) | 4.0 ± 0.8 | 3.7 ± 0.7 | 3.9 ± 0.5 | 3.7 ± 0.6 | 3.8 ± 0.6 | 4.6 ± 1.0 | <0.001 |
| Uric acid (mg/dl) | 7.3 ± 1.9 | 6.4 ± 2.0 | 5.5 ± 2.1 | 7.4 ± 2.0 | 7.7 ± 1.6 | 7.6 ± 1.5 | 0.004 |
| Cholesterol (mg/dl) | 176 ± 55 | 178 ± 53 | 209 ± 103 | 173 ± 51 | 173 ± 45 | 170 ± 47 | |
| Albumin (g/dl) | 3.7 ± 0.6 | 4.0 ± 0.8 | 3.7 ± 0.6 | 3.7 ± 0.6 | 3.8 ± 0.6 | 3.5 ± 0.5 | 0.023 |
| Urea (mg/dl) | 35.9 ± 15.4 | 15.0 ± 3.2 | 22.6 ± 6.4 | 31.1 ± 14.4 | 36.6 ± 9.7 | 48.8 ± 13.8 | <0.001 |
| Creatinine (mg/dl) | 3.09 ± 2.40 | 0.79 ± 0.11 | 1.01 ± 0.16 | 1.68 ± 0.30 | 2.64 ± 0.53 | 6.01 ± 2.55 | <0.001 |
| eGFR (mg/min/1.73 m²) | 33.9 ± 28.1 | 107.4 ± 13.7 | 75.6 ± 9.2 | 40.4 ± 7.5 | 22.2 ± 4.1 | 9.6 ± 3.0 | <0.001 |
| Urine PCR 9 g/g) | 3.8 ± 4.6 | 2.0 ± 3.2 | 2.4 ± 4.1 | 3.2 ± 5.0 | 2.8 ± 3.5 | 6.1 ± 4.7 | <0.001 |

The median of ages was 61.3, 138 patients were men. The serum creatinine was 3.09±2.40 mg/dl and, when calculated by the CKD-EPI creatinine equation, the estimated glomerular filtration rate (GFR) was 33.9±28.1 ml/min/1.73 m².

As CKD progressed from stage I to stage V, it could be seen that UPCR, urine cMet and urine cMet/Cr levels were increased.

### Example 2—Measurement of Urine cMet Level by ELISA

Soluble cMet ELISA (Thermo Fisher Scientific Inc. (Waltham, Mass., USA), Lot Nos: 1696848A2 and 1877133A) was executed according to instruction of the manufacturer. A lower Limit of Quantification (LLOQ) of the soluble cMet was 0.78 ng/ml. Significant matrix effects were not demonstrated in human urine samples.

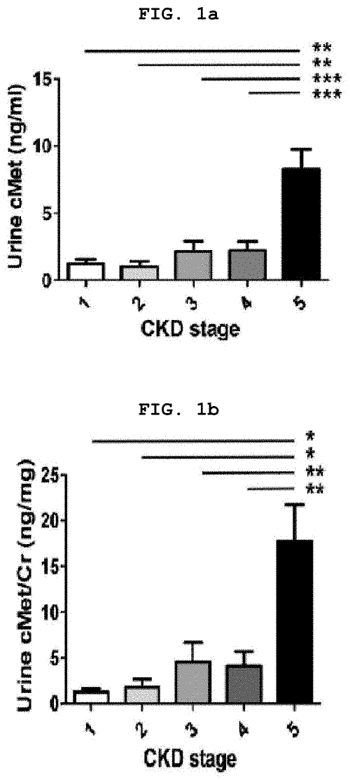

The soluble urine cMet level measured by ELISA was significantly high in patients with chronic nephropathy stage V, as compared to patients with chronic nephropathy stages I, II, III or IV (P<0.001). Such tendency was not changed even after adjusting the soluble urine cMet level based on urine creatinine (see FIGS. 1A, 1B and Table 1).

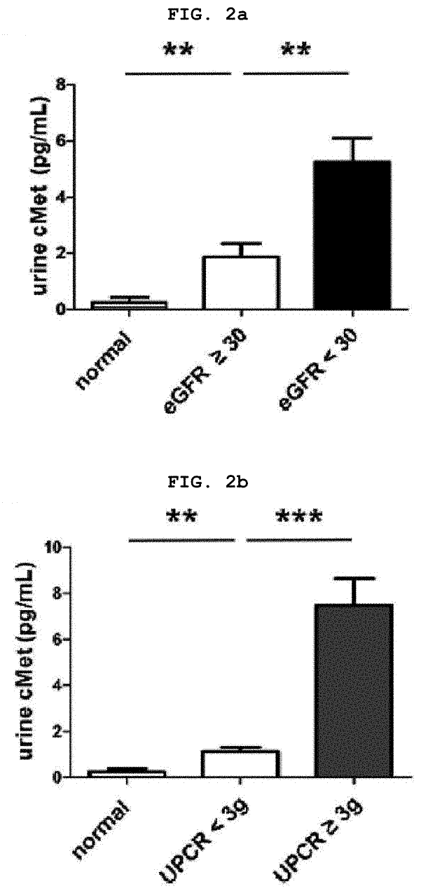

Further, as compared to patients with relatively retained kidney functions (1.86±4.77 ng/ml, P=0.001) as well as eGFR of 30 ml/min/1.73 m² or more, patients with deteriorated kidney functions (5.25±9.62 ng/ml, P=0.001) as well as eGFR of less than 30 ml/min/1.73 m² exhibited higher urine cMet level (FIG. 2A). Further, the urine cMet level was higher in patients with nephritic proteinuria (7.58±11.28 ng/ml, P<0.001), than patients with sub-nephrotic proteinuria (1.15±2.28 ng/ml, P<0.001) (FIG. 2B).

Further, the serum and urine samples obtained from patients with minimal change disease (MCD), focal segmental glomerulosclerosis (FSGS), IgA nephropathy (IgAN) and diabetic nephropathy (DN), respectively, were subjected to measurement of cMet concentration (Table 2). The values in separate categories in Table 2 were represented in the median (minimum, maximum) mode, and divided into two parts when blood test at the time of kidney biopsy was conducted and after appropriate treatment has proceeded in separate diseases, respectively.

TABLE 2

| | MCD | | FSGS | | IgAN | | DMN | |
|---|---|---|---|---|---|---|---|---|
| | (n = 7) | | (n = 6) | | (n = 35) | | (n = 218) | |
| Male gender | 2 | (28.6) | 4 | (66.7) | 23 | (62.2) | 138 | (63.3) |
| (n, %) | | | | | | | | |
| Age (years) | 58.0 | (43.0, 70.0) | 59.0 | (38.3, 72.5) | 41.0 | (27.5, 54.0) | 61.3 | (51.0, 70.0) |

Blood test at the time of kidney biopsy

| | | | | | | | | |
|---|---|---|---|---|---|---|---|---|
| BUN (mg/dl) | 13.0 | (8.0, 66.0) | 20.0 | (9.5, 30.5) | 15.0 | (10.3, 23.8) | 35.9 | (20.4, 50.1) |
| Serum creatinine | 0.87 | (0.55, 2.91) | 1.29 | (0.80, 1.43) | 1.16 | (0.75, 1.64) | 3.09 | (1.57, 4.36) |
| (mg/dl) | | | | | | | | |
| eGFR_ | 85.9 | (16.1, 115.2) | 58.1 | (39.1, 104.2) | 58.3 | (41.8, 103.6) | 32.9 | (16.2, 41.9) |
| Urine protein/ | 15.59 | (6.1, 21.25) | 8.27 | (3.47, 10.61) | 1.64 | (0.92, 3.59) | 3.74 | (0.61, 6.02) |
| creatinine ratio | | | | | | | | |
| (mg/mgCr) | | | | | | | | |
| Serum cMet | 0.93 | (0.76, 1.13) | 0.75 | (0.62, 0.82) | 0.41 | (1.12, 2.47) | 3.8 | (0.00, 3.06) |
| level(ng/ml) | | | | | | | | |
| Urine cMet/ | 0.043 | (0.032, 0.066) | 0.022 | (0.014, 0.071) | 01010 | (0.004, 0.022) | 0.004 | (0.000, 0.041) |
| creatinine ratio | | | | | | | | |
| (ng/mgCr) | | | | | | | | |

Blood test at follow up

| | | | | | | | | |
|---|---|---|---|---|---|---|---|---|
| BUN | 15.0 | (10.0, 22.0) | 25.5 | (13.8, 36.5) | 20.0 | (12.0, 24.8) | 42.7 | (25.1, 59.2) |
| (mg/dl) | | | | | | | | |
| Serum | 0.72 | (0.57, 0.84) | 1.60 | (1.01, 1.84) | 1.17 | (0.86,1.80) | 3.67 | (1.49, 6.15) |
| creatinine | | | | | | | | |
| (mg/dl) | | | | | | | | |
| eGFR (mg/min/ | 103.0 | (79.0, 115.3) | 46.4 | (34.3, 71.2) | 61.4 | (37.7, 91.7) | 16.0 | (8.71, 40.2) |
| 1.73 m²) | | | | | | | | |
| Urine protein/ | 0.08 | (0.05, 0.13) | 4.52 | (0.95, 11.29) | 0.71 | (0.20, 1.25) | 2.61 | (1.02, 8.73) |
| creatinine ratio | | | | | | | | |
| (mg/mgCr) | | | | | | | | |

### Example 3—Relationship Between Soluble Urine cMet and Clinical Result

The clinical results used herein are ESRD, all-cause mortality and a combination thereof. ESRD was defined as a non-reversible damage of kidney functions which requires kidney dialysis or kidney transplant. Mortality data of the patients without continuous tracking inspection was acquired from the database of The National Statistical Office of Korea. During tracking observation for 49.8±19.6 months, 101 patients (46.1%) were diagnosed as ESRD while 65 patients (29.7%) were dead. In order to identify relationship between soluble urine cMet concentration and ESRD or the death, Kaplan-Meier survival assay was executed.

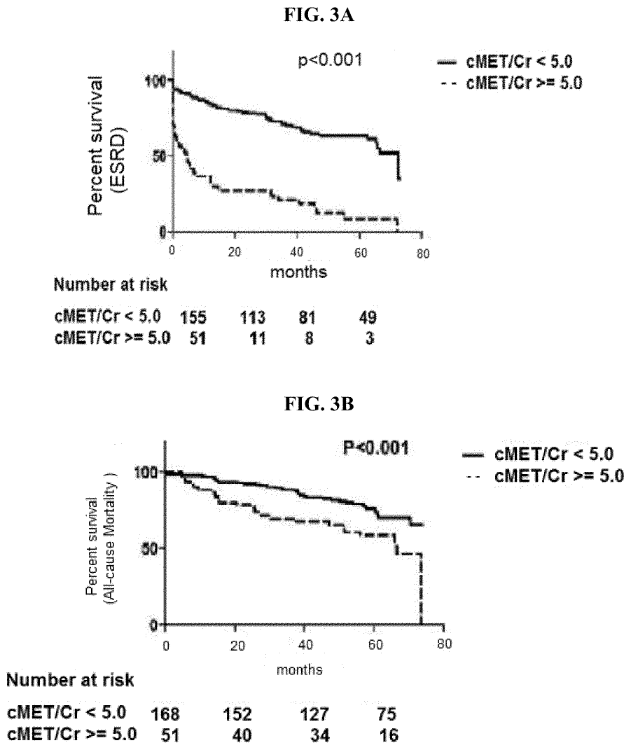

As compared DN patients having lower urine cMet/Cr level, DN patients having higher urine cMet/C level showed higher ESRD risk (FIG. 3A), all-caused mortality (FIG. 3B) and combined assessment results (FIG. 3C) (P<0.001). Table 3 below shows relationship between soluble urine cMet level, transplant failure and mortality, which were verified using Cox proportional hazards model.

TABLE 3

| Unadjusted | | Model 1 | | Model 2 | |
|---|---|---|---|---|---|
| HR(95% CI) | P | HR(95% CI) | P | HR(95% CI) | P |

ESRD
cMET

| | | | | | | |
|---|---|---|---|---|---|---|
| Cut-off value | 4.39 (2.92-6.59) | <0.001 | 3.75 (2.17-6.48) | <0.001 | 2.33 (1.19-4.57) | 0.014 |

cMEt/Cr

| | | | | | | |
|---|---|---|---|---|---|---|
| Cut-off value | 5.04 (3.35-7.59) | <0.001 | 4.73 (2.67-8.36) | <0.001 | 3.07 (1.66-5.69) | 0.001 |

Composite
cMet

| | | | | | | |
|---|---|---|---|---|---|---|
| Cut-off value | 3.71 (2.59-5.32) | <0.001 | 3.17 (1.94-5.19) | <0.001 | 2.14 (1.18-3.86) | 0.012 |

cMet/Cr

| | | | | | | |
|---|---|---|---|---|---|---|
| Cut-off value | 4.16 (2.89-6.00) | <0.001 | 3.41 (2.02-5.76) | <0.001 | 2.11 (1.20-3.71) | 0.009 |

Mortality
cMet

| | | | | | | |
|---|---|---|---|---|---|---|
| Cut-off value | 2.13 (1.29-3.52) | 0.003 | 2.42 (1.00-5.84) | 0.05 | 1.88 (0.81-4.39) | 0.142 |

cMet/Cr

| | | | | | | |
|---|---|---|---|---|---|---|
| Cut-off value | 2.07 (1.24-3.45) | 0.005 | 2.42 (1.09-5.38) | 0.031 | 1.96 (0.86-4.46) | 0.11 |

Table 3 above shows relationship between soluble urine cMet level, transplant failure and mortality, using conventional Cox proportional hazards model. In this regard, model 1 was adjusted with respect to age, gender, hypertension, Hb, albumin, AST, ALT, uric acid, cholesterol, Ca and P, while model 2 was adjusted with respect to age, gender, hypertension, Hb, albumin, AST, ALT, uric acid, cholesterol, Ca, P, GFR and PCR. Analysis was performed with calculation including predetermined cutoff values of the soluble urine cMet and cMet/Cr levels in ROC curve. The cutoff values with respect to mortality and ESRD were 2.9 ng/ml and 4.9 ng/mg, respectively. HR means a hazard ratio and CI denotes a confidence interval. Increased cMet value was maintained in a case of combined assessment results (HR: 2.14, 95% CI: 1.18 to 3.86, P=0.012) and mortality (HR: 1.88, 95% CI: 0.81 to 4.39, P=0.142) after application of independent variables (hazard ratio (HR): 2.33, 95% confidence interval (CI): 1.19 to 4.57, P=0.014) relevant to ESRD or confounding variables such as age, gender, hemoglobin, albumin, AST, ALT, uric acid, cholesterol, Ca, P, estimated GFR (eGFR) and UPCR.

### Example 4—Soluble Urine cMet/Cr with Respect to Presumption of Negative Result

Whether the soluble urine cMet/Cr level is a better index for ESRD than the serum creatinine and UPCR, was identified by comparing ROC curve, integrated discrimination improvement (IDI) index and category-free net reclassification improvement (cfNRI) index. AUC (95% CI) of Cr (model 1), Cr+UPCR (model 2) and Cr+UPCR+cMet (model 3) were 0.858 (0.808-0.907), 0.889 (0.846-0.931) and 0.890 (0.848-0.933), respectively. Adding the soluble urine cMet/Cr level to serum Cr or serum Cr+UPCR has statistically improved estimated values with respect to ESRD and the combined assessment results except for mortality (Table 4).

TABLE 4

| | DeLong | | | | |
|---|---|---|---|---|---|
| AUC | test | IDI | | Category-free NRI | |
| (95% CI) | P-value | P-value | (95% CI) | P-value | (95% CI) |

ESRD

| | | | | | | |
|---|---|---|---|---|---|---|
| Cr | 0.858 | Reference | Reference | | Reference | |
| | (0.808, | | | | | |
| | 0.907) | | | | | |
| cMet/Cr | 0.694 | <0.0001 | <0.0001 | | <0.0001 | |
| | (0.623, | | | | | |
| | 0.765) | | | | | |
| Cr + UPCR | 0.889 | 0.0389 | 0.0001 | 6.52% | <0.0001 | 88.31% |
| | (0.846, | | | (3.18%, | | (65.02%, |
| | 0.931) | | | 9.87%) | | 111.61%) |
| Cr + UPCR + cMet/Cr | 0.890 | 0.0295 | 0.0001 | 6.76% | <0.0001 | 82.64% |
| | (0.848, | | | (3.38%, | | (59.07%, |
| | 0.933) | | | 10.15%) | | 106.22%) |

Death

| | | | | | | |
|---|---|---|---|---|---|---|
| Cr | 0.644 | Reference | Reference | | Reference | |
| | (0.569, | | | | | |
| | 0.720) | | | | | |
| cMET/Cr | 0.620 | 0.5943 | 0.0748 | | 0.1726 | |
| | (0.536, | | | | | |
| | 0.704) | | | | | |
| Cr + UPCR | 0.633 | 0.5715 | 0.4641 | 0.32% | 0.3923 | 11.95% |
| | (0.556, | | | (−0.53%, | | (−15.42%, |
| | 0.710) | | | 1.17%) | | 39.31%) |
| Cr + UPCR + cMet/Cr | 0.641 | 0.9138 | 0.0372 | 2.76% | 0.8453 | −2.82% |
| | (0.558, | | | (0.16%, | | (−31.11%, |
| | 0.724) | | | 5.35%) | | 25.47%) |

Composite

| | | | | | | |
|---|---|---|---|---|---|---|
| Cr | 0.865 | Reference | Reference | | Reference | |
| | (0.818, | | | | | |
| | 0.911) | | | | | |
| cMet/Cr | 0.694 | <0.0001 | <0.0001 | | <0.0001 | |
| | (0.626, | | | | | |
| | 0.761) | | | | | |
| Cr + UPCR | 0.878 | 0.149 | 0.0196 | 2.86% | <0.0001 | 81.06% |
| | (0.835, | | | (0.46%, | | (57.53%, |
| | 0.922) | | | 5.27%) | | 104.59%) |
| Cr + UPCR + cMet/Cr | 0.886 | 0.0555 | 0.0005 | 4.78% | <0.0001 | 74.21% |
| | (0.844, | | | (2.10%, | | (50.30%, |
| | 0.928) | | | 7.46%) | | 98.12%) |

IDI and cfNRI (model 1 vs. model 3) for ESRD estimation were 6.76% (95% CI: 3.38-10.15, P=0.0001) and 82.64% (95% CI: 59.07-106.22, P<0.0001), respectively. It could be seen that measuring the soluble urine cMet level increased the estimated value for substantially negative results. In the above table, Cr is creatinine, UPCR is urine protein-to-creatinine ratio, CI is confidence interval, IDI is integrated discriminatory improvement, and NRI is net reclassification improvement.

### Example 5—Identification of cMet Expression Pattern Through Histological Inspection of Patient-Derived Kidney

Among the kidney excised from a patient with renal cancer by kidney whole resection, a non-tumor tissue portion was fixed with 4% para-formaldehyde, followed by embedding the same using paraffin, cutting into a size of 4 μm and then staining the same. After removing paraffin from this section in xylene, and the section was gradually re-hydrated with a series of ethanol. Activation of endogenous peroxidase was blocked with 0.3% hydrogen peroxidase in methanol at room temperature for 30 minutes. The section was subjected to microwave treatment with an antigen-unmasking solution for 30 minutes to recover antigen. After incubating the section in 10% goat serum at room temperature for 1 hour, the product was reacted with anti-Met (cMet) antibody (Abcam, Cambridge, UK) and anti-Met (cMet) (phosphor Y1349) antibody (Abcam) at 4° C. overnight. The tissue was washed with phosphate-buffered saline (PBS) several times, and cultured with Alexa Fluor-fused secondary antibody (Molecular, Probes, Eugene, Oreg.) for 40 minutes. A nucleus was contrast-stained using DAPI (4′, Molecular Probes). A negative control section was treated in the same manner as described above, except that primary antibodies were omitted. The section was observed through Leica TCS SP8 STED CW confocal microscope.

From healthy human glomerular cells, podocytes, mesangial cells and proximal tubular epithelial cells (PTEC), fluorescence to each of DAPI (blue), cMet (green), and CD31, nephrin, desmin or AQP1 (red) was detected (FIG. 4A).

According to immunofluorescence staining using a phosphorylation-specific cMet antibody to identify activated cMet, it has been verified whether HGF/Met route was associated with renal fibrosis in DN patient. Phosphorylated cMet level was higher in the glomerulus of the DN patient than a normal control. Based on the results, it could be seen that cMet was activated in the DN patient (FIG. 4B).

### Example 6—Relationship Between IgA Nephropathy and Soluble Urine cMet Level

104 IgA nephropathy patients were divided into three groups based on UPCR level (UPCR 1.0; 1.0<UPCR<3.5; and UPCR 3.5). As a result of tracking observation for 35 months, average ages of these groups were 33.0, 41.0 and 55.5. As UPCR level is increased, cMet/Cr level tended to be higher. Further, for patients with higher cMet/Cr level, they increasingly progressed to ESRD, and poor prognosis was demonstrated (Table 5).

TABLE 5

| | Group 1 | | Group 2 | | Group 3 | | |
|---|---|---|---|---|---|---|---|
| | (n = 33) | | (n = 43) | | (n = 28) | | |
| | (UPCR = 1.0) | | (1.0 < UPCR < 3.5) | | (UPCR = 3.5) | | P value |
| Age | 33.0 | (19.5, 45.0) | 41.0 | (30.0, 52.0) | 55.5 | (37.3, 70.0) | <0.001 |
| Male gender | 26 | (78.8) | 23 | (53.5) | 12 | (42.9) | 0.012 |
| Smoking history | 4 | (12.1) | 11 | (25.6) | 7 | (25.0) | 0.306 |
| Diabetes | 0 | (0.0) | 0 | (0.0) | 3 | (10.7) | 0.016 |
| Hypertension | 14 | (42.4) | 29 | (67.4) | 24 | (85.7) | 0.002 |
| SBP | 127.0 | (116.5, 138.5) | 135.0 | (123.0, 149.0) | 140.0 | (131.5, 150.0) | 0.002 |
| DBP | 79.0 | (71.5, 85.5) | 86.0 | (79.0, 97.0) | 92.0 | (81.5, 97.8) | 0.002 |
| BMI | 23.8 | (21.4, 26.0) | 23.8 | (21.7, 26.1) | 24.4 | (21.3, 26.4) | 0.563 |
| Microscopic hematuria | 29 | (87.9) | 38 | (88.4) | 25 | (89.3) | 0.866 |
| SMK grade | | | | | | | <0.001 |
| I | 4 | (13.3) | 0 | (0.0) | 0 | (0.0) | |
| II | 19 | (63.3) | 13 | (38.2) | 5 | (21.7) | |
| III | 7 | (23.3) | 13 | (38.2) | 7 | (30.4) | |
| IV | 0 | (0.0) | 5 | (14.7) | 7 | (30.4) | |
| V | 0 | (0.0) | 3 | (8.8) | 4 | (17.4) | |
| Haas Class | | | | | | | 0.001 |
| I | 3 | (10.0) | 0 | (0.0) | 0 | (0.0) | |
| II | 3 | (10.0) | 0 | (0.0) | 2 | (8.7) | |
| III | 17 | (56.7) | 13 | (38.2) | 3 | (13.0) | |
| IV | 6 | (20.0) | 15 | (44.1) | 10 | (43.5) | |
| V | 0 | (0.0) | 6 | (17.6) | 8 | (34.8) | |
| VI | 1 | (3.3) | 0 | (0.0) | 0 | (0.0) | |
| Mesangial hypercellularity | 32 | (97.0) | 37 | (86.0) | 27 | (96.4) | 0.854 |
| Interstitial fibrosis/ | | | | | | | |
| tubularatrophy | | | | | | | |
| Moderate to severe | 4 | (12.1) | 10 | (23.3) | 13 | (50.0) | 0.014 |
| Interstitial inflammation | | | | | | | |
| Moderate to severe | 2 | (6.2) | 8 | (18.6) | 11 | (42.3) | 0.003 |
| Vessel | | | | | | | |
| Fibrointimal thickening | 8 | (24.2) | 17 | (39.5) | 18 | (64.3) | 0.006 |
| Hyaline arteriolosclerosis | 1 | (3.0) | 9 | (20.9) | 9 | (32.1) | 0.011 |
| Global sclerosis (%) | 5.6 | (0.0, 15.2) | 25.0 | (3.3, 50.0) | 32.9 | (17.2, 63.2) | <0.001 |
| Segmental sclerosis (%) | 0.0 | (0.0, 7.0) | 4.3 | (0.0, 12.0) | 9.7 | (0.0, 16.6) | 0.006 |
| Crescent (%) | 0.0 | (0.0, 0.0) | 0.0 | (0.0, 0.0) | 0.0 | (0.0, 2.4) | 0.054 |
| sCr_baseline | 0.85 | (0.74, 1.00) | 1.10 | (0.79, 1.60) | 1.50 | (1.05, 2.25) | <0.001 |
| Egfr_baseline | 107.3 | (81.2, 117.3) | 62.2 | (46.1, 87.6) | 42.0 | (26.4, 61.7) | <0.001 |
| UPCR_baseline | 0.31 | (0.17, 0.69) | 2.02 | (1.40, 2.57) | 4.64 | (3.97, 6.13) | <0.001 |
| IgA_baseline | 354.5 | (245.3, 441.8) | 340.0 | (276.5, 441.3) | 346.0 | (251.0, 462.0) | 0.755 |
| Albumin_baseli ne | 4.1 | (3.8, 4.2) | 3.7 | (3.4, 4.0) | 3.3 | (2.9, 3.5) | <0.001 |
| hsCRP_baseline | 0.16 | (0.04, 0.34) | 0.12 | (0.07, 0.40) | 0.31 | (0.10, 0.58) | 0.048 |
| T.chol_baseline | 162.0 | (146.5, 183.5) | 173.0 | (155.0, 205.0) | 212.0 | (194.3, 229.8) | <0.001 |
| Uric acid_baseline | 6.0 | (4.7, 6.8) | 6.5 | (5.2, 7.6) | 6.9 | (5.7, 8.1) | 0.021 |
| Urine cMet | 0.94 | (0.26, 1.73) | 0.94 | (0.32, 1.97) | 1.97 | (0.68, 4.04) | 0.008 |
| Urine cMet/Cr | 0.007 | (0.003, 0.018) | 0.015 | (0.003, 0.027) | 0.030 | (0.012, 0.060) | <0.001 |
| Ln_urinecMet/Cr | −4.9 | (−5.9, −4.1) | −4.1 | (−5.7, −3.6) | −3.5 | (−4.4, −2.8) | <0.001 |
| Treated with RAS blockade | 21 | (63.3) | 34 | (79.1) | 26 | (92.9) | 0.023 |
| Treated with statin | 6 | (18.2) | 19 | (44.2) | 14 | (50.0) | 0.019 |
| Treated with immuno | 1 | (3.0) | 11 | (25.6) | 13 | (46.4) | <0.001 |
| suppressive agents | | | | | | | |
| eGFR_final | 100.7 | (83.0, 113.3) | 59.0 | (33.9, 100.4) | 36.7 | (12.7, 48.8) | <0.001 |
| UPCR_final | 0.28 | (0.09, 0.60) | 0.95 | (0.51, 1.75) | 1.24 | (0.31, 1.76) | 0.005 |
| Complete remission | 17 | (51.5) | 7 | (16.3) | 5 | (17.9) | 0.001 |
| Progression to ESRD | 0 | (0.0) | 5 | (11.6) | 9 | (32.1) | 0.001 |

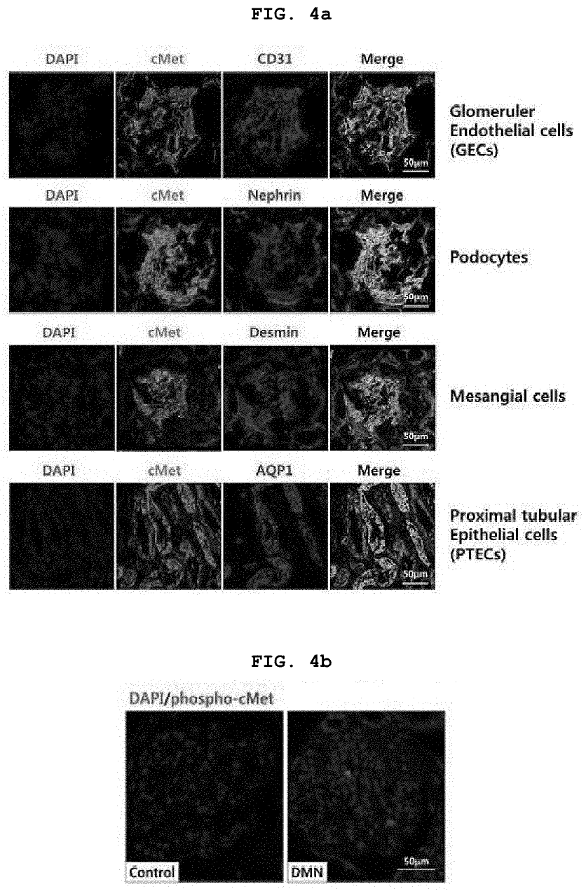

Further, it could be demonstrated from KM curve that, as IgA nephropathy patients with higher soluble urine cMet concentration are increased, a ratio of patients showing 50% increase of proteinuria is higher (Kaplan, E. L.; Meier, P. (1958). “Nonparametric estimation from incomplete observations”. J. Amer. Statist. Assoc. 53 (282): 457-481. doi:10.2307/2281868. JSTOR 2281868; Kaplan, E. L. in a retrospective on the seminal paper in “This week's citation classic”. Current Contents 24, 14 (1983).) (FIG. 5). Variables used in KM curve are shown in Table 6 below.

TABLE 6

| | | | | | | | 95.0% CI for Exp(B) | |
|---|---|---|---|---|---|---|---|---|
| | B | SE | Wald | df | Sig. | Exp(B) | Lower | Upper |
| Ln_UcMet_Cr | −.365 | .179 | 4.160 | 1 | .041 | .694 | .489 | .986 |

The inventors of the present invention firstly discovered a relationship between increase in soluble urine cMet level and kidney functions in patients with renal disease. In particular, it was found that the increase in soluble urine cMet level is significantly relevant to survival of the kidney and mortality. In a completely adapted ESRD model, inclusion of the soluble urine cMet level could improve estimation ability in three types of statistical methods (C-statistics, IDI and NRI). Accordingly, in the case of DN, it is understood that the soluble urine cMet level may be used as a novel biomarker for predicting prognosis of the kidney during diagnosis.

### Example 7—Measurement of cMet Concentration in Urine by Administration of Anti-cMet Antibody in Acute Kidney Disease (AKD) Mouse Model

In order to prepare an acute kidney disease mouse model, after general anesthesia of mouse, a side abdomen of the mouse was incised to expose the pedicle of a kidney and tie blood vessels using a microvascular clamp, followed by reperfusion through the blood vessels after 27 minutes. During surgical operation, the body temperature was maintained at 37° C. on a temperature-controlled operating table while preventing water loss was prevented. The control group (sham) was subjected to the same treatment as described above to expose the pedicle of a kidney except that the mouse without induction of the blood vessel clamping was used. The examination was implemented for separate groups including, for example, a saline administered control mouse (n=1), an antibody administered control mouse (n=1), saline administered AKD mice (n=3), and anti-cMet antibody administered AKD mice (n=6). The antibody used herein is sequences (VH0, Vk0) prepared by substituting a lysine residue (K) in CH1 domain among previously developed anti-cMet antibodies 1E4 (WO2016/021864A1, and WO2017/135791A1) with an arginine residue (R) to convert the antibody, thereby completely matching with Glm(3) allotype; and substituting a serine residue (S) in the light chain constant region with a threonine residue (T) to convert the antibody, thereby completely matching with Km3 allotype. Such sequences are shown in Table 7 below.

Further, in order to verify biological activity of VH0/Vk0 in in vitro level, HUVEC migration assay was performed. As a result of the experiment, similar to a positive control group including hepatocyte growth factor (HGF), both of 1E4 and VH0/Vk0 exhibited effects of accelerating HUVEC migration at a concentration of 90 ng/ml. In a quantification aspect, 1E4 and VH0/Vk0 could accelerate cell migration in very similar levels of 158% and 161%, respectively, as compared to the negative control group. From the above results, it could be seen that existing biological activity was retained regardless of the altered corresponding constant region. Further, it is presumed that, if altering the constant region to be coincident with human genital cell-relevant sequence, in vivo stability (e.g., immunogenicity) and safety (e.g., half-life) may be enhanced though reduction in immunogenicity and neutralization (Reference: Lefranc, M.-P., and Lefranc, G. (2012). Human Gm, Km and Am allotypes and their molecular characterization: a remarkable demonstration of polymorphism. In immunogenetics (F. Christiansen and B. Tait, Eds.), Chap. 34. Humana Press, Springer, New York. Methods Mol Biol. 882,635-680.)

With reference to day 0 (D0) of surgical operation, anti-cMet antibody (Table 7) was intravenously administered (20 mg/kg) on −D1, D0, D3, D7, D10, D14 and D21, respectively. At 4 weeks after the operation, urine was collected from the mouse and subjected to measurement of urine cMet concentration by Mouse Hepatocyte Growth Factor Receptor (CMET/HGFR) ELISA Kit (Catalog No. MBS2019020, MyBioSource; San Diego, Calif., USA).

TABLE 7

| SEQ ID | | |
|---|---|---|
| NOs | Description | Amino acid sequence |
| 1 | HCDR1 | THWIT |
| 2 | HCDR2 | TIDPTDSYNFYGPSFQG |
| 3 | HCDR3 | DGNYYDSRGYYYDTFDM |
| 4 | LCDR1 | RASQGISTYLA |
| 5 | LCDR2 | SASTLES |
| 6 | LCDR3 | QQADSFPLT |
| 7 | Heavy chain | QVQLVQSGAEVKKPGESLRISCQGSGYSFPTHW |
| | variable | ITWVRQMPGKGLEWMGTIDPTDSYNFYGPSFQG |
| | region | HVTISADSSSSTAYLQWSSLKASDTAMYYCARD |
| | (VH0) | GNYYDSRGYYYDTFDMWGQGTLVTVSS |
| 8 | Light chain | DIQMTQSPSFLSASVGDRVTITCRASQGISTYL |
| | variable | AWYQQKPGTAPKLLIYSASTLESGVPSRFSGSG |
| | region | SGTDFTLTISSLQPEDSATYYCQQADSFPLTFG |
| | (Vk0) | GGTKVEIK |

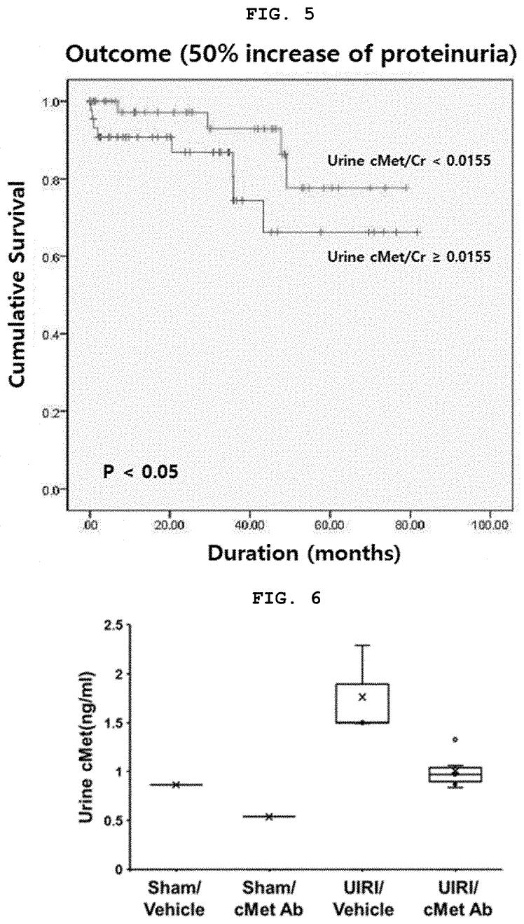

As a result, it was demonstrated that the mouse having derived AKD has about 2-fold increased urine cMet concentration. On the other hand, it could be seen that, when treated with anti-cMet antibody, the urine cMet concentration was decreased to a similar level to that of a normal mouse (Table 8, and FIG. 6). Referring to Table 8 below, the rows of each column indicates values measured from individuals in the same experimental group.

TABLE 8

| | | Sham/cMet | | |
|---|---|---|---|---|
| Label | Sham/Vehicle | Ab | UIRI/Vehicle | UIRI/cMet |
| cMet | 0.863 | 0.538 | 1.5015 | 1.3285 |
| (ng/ml) | | | 1.494 | 0.8375 |
| | | | 2.29 | 1.065 |
| | | | | 0.87 |
| | | | | 0.97 |
| | | | | 0.97 |

UIRI: Unilateral Ischemia-Reperfusion Injury

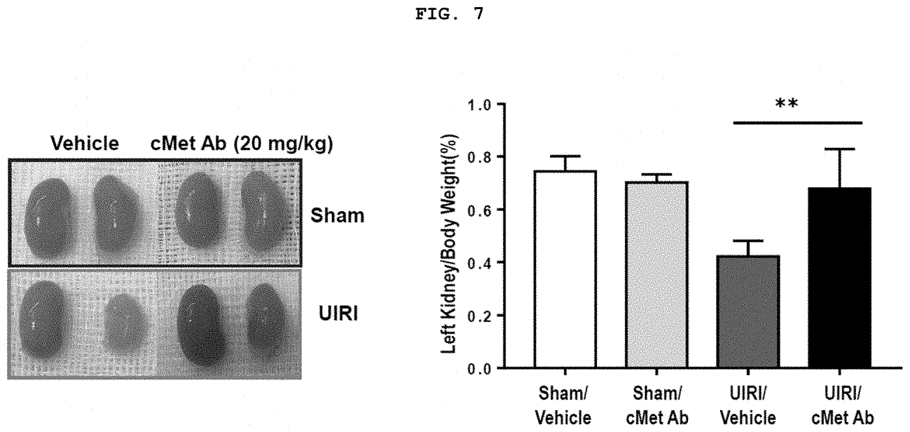

Further, the mouse model was sacrificed at day 28, followed by isolating the kidney, and then, a size and a mass of the kidney were compared to the body weight (FIG. 7). It was demonstrated that, even though the anti-cMet antibody was administered to Sham group, the size and the mass of the kidney were not influenced by the same. For UIRI model, the size and mass of the kidney were decreased as compared to sham. However, in the case of administering the anti-cMet antibody to UIRI model, the size and mass of the kidney were substantially the same as the sham group.

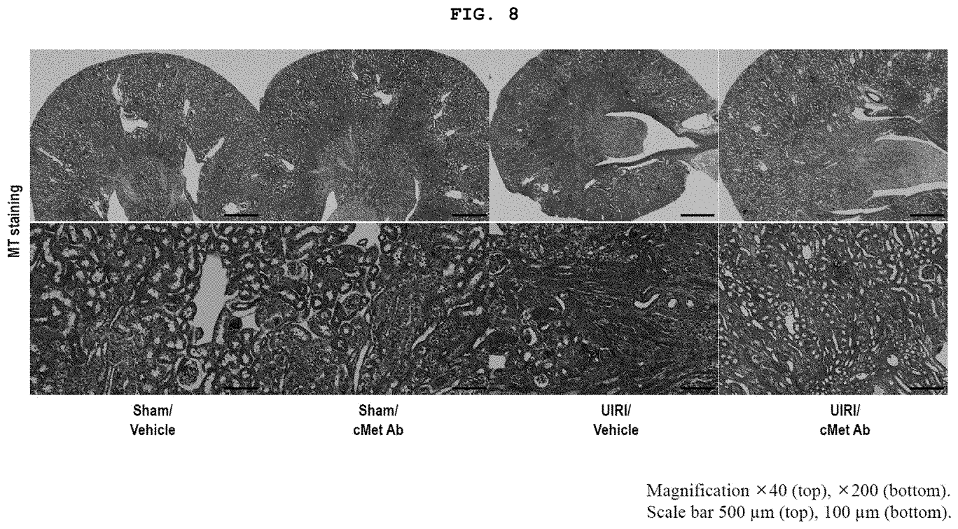

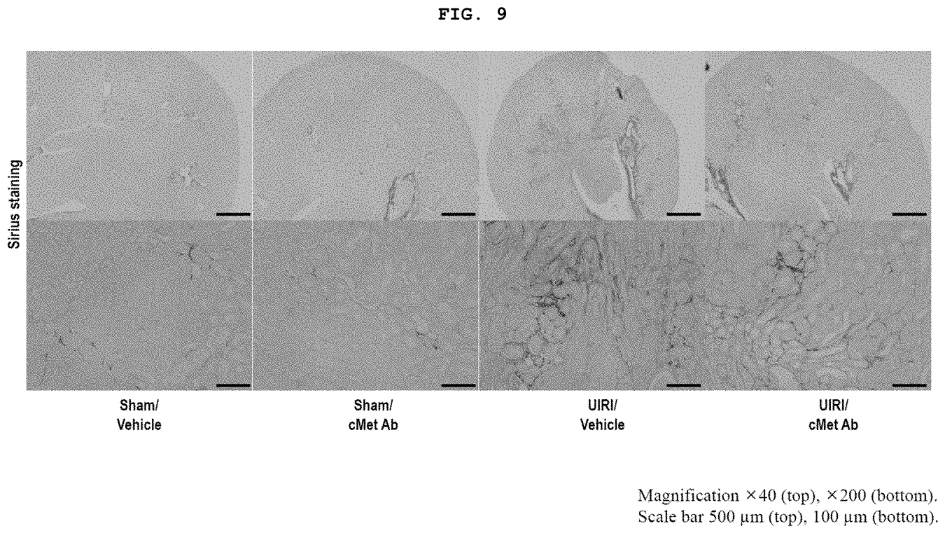

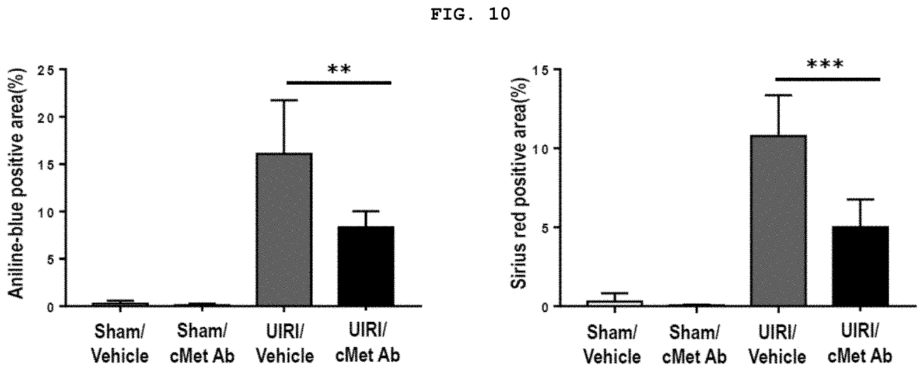

Further, as a result of inspecting the kidney tissue through MT staining, UIRI model showed progress of kidney fibrosis (FIG. 8, and the left side of FIG. 10). This phenomenon was substantially coincided with the results of Sirius staining (FIG. 9, and the right side of FIG. 10). On the other hand, when the anti-cMet antibody was administered to the UIRI model, significantly reduced fibrosis was demonstrated (FIGS. 8, 9 and 10).

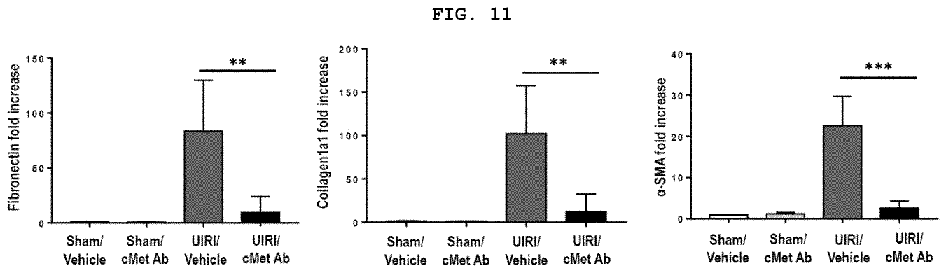

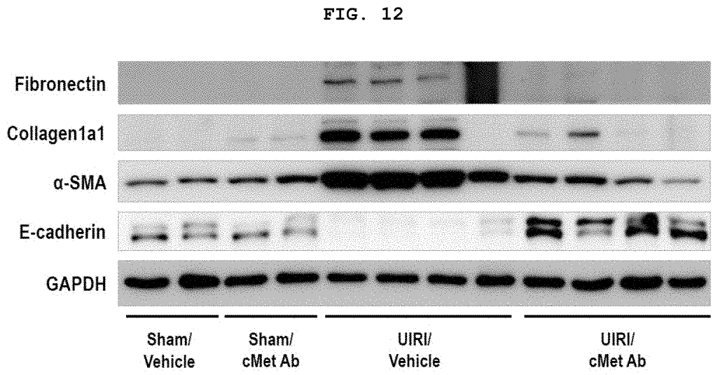

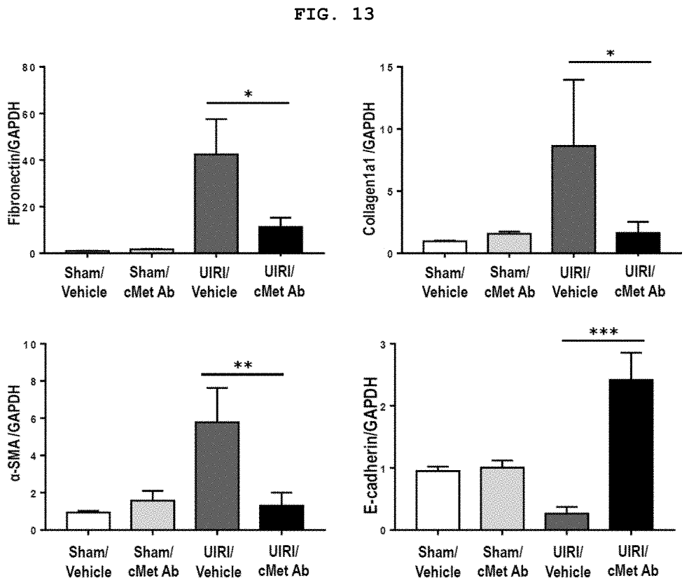

In order to identify kidney fibrosis in an aspect of molecular biology, RT-PCR was performed with respect to molecular indicators of fibrosis such as fibronectin, collagen1 and αSMA. As a result, it could be seen that these three indicators were remarkably expressed in the UIRI model, whereas, when the anti-cMet antibody was administered to the UIRI model, expression thereof was decreased to a level similar to that of the normal group (FIG. 11). Further, such tendency was also verified through Western blotting (FIGS. 12 and 13).

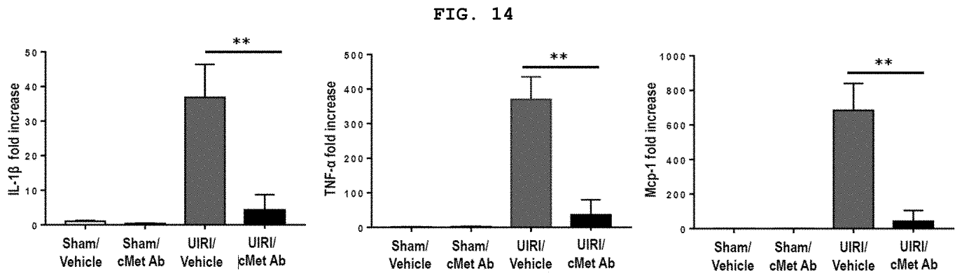

Meanwhile, as a result of inspecting expression levels of inflammatory factors, i.e., IL-1β, TNF-α and Mcp-1, it was observed that UIRI model exhibited remarkably increased expression of IL-1β, TNF-α and Mcp-1, whereas, when the anti-cMet antibody was administered to the UIRI model, the expression levels of IL-1β, TNF-α and Mcp-1 were substantially similar to that of the normal group (FIG. 14).

### Example 8—Improvement of Stability of VH0/Vk0 Antibody

In order to enhance stability of VH0/Vk0 antibodies, the present inventors executed CDR grafting using a mother antibody 1E4 and thus prepared VH4, VH5, VH6 and Vk2 (Table 9) (PCT/KR2019/015475).

TABLE 9

| SEQ | | |
|---|---|---|
| ID | | |
| NOs | Description | Amino acid sequence |
| 9 | Heavy | QVQLVQSGAEVKKPGSSVKVSCQGSGYSFPTH |
| | chain | WITWVRQAPGQGLEWMGTIDPTDSYNFYGPSF |
| | variable | QGRVTITADSSTSTAYMELSSLRSEDTAMYYC |
| | region | ARDGNYYDSRGYYYDTFDMWGQGTLVTVSS |
| | (VH4) | |
| 10 | Heavy | QVQLVQSGAEVKKPGSSVKVSCQGSGYSFPTH |
| | chain | WITWVRQAPGQGLEWMGTIDPTDSYNFYGPSF |
| | variable | QGRVTITADSSTSTAYMELSSLRSEDTAVYYC |
| | region | ARDGNYYDSRGYYYDTFDMWGQGTLVTVSS |
| | (VH5) | |
| 11 | Heavy | QVQLVQSGAEVKKPGSSVKVSCKASGYSFPTH |
| | chain | WITWVRQAPGQGLEWMGTIDPTDSYNFYGPSF |
| | variable | QGRVTITADKSTSTAYMELSSLRSEDTAVYYC |
| | region | ARDGNYYDSRGYYYDTFDMWGQGTLVTVSS |
| | (VH6) | |
| 12 | Light | DIQMTQSPSSLSASVGDRVTITCRASQGISTY |
| | chain | LAWYQQKPGKAPKLLIYSASTLESGVFPSRFS |
| | variable | GSGSGTDFTLTISSLQPEDATYYCQQADSFPL |
| | region | TFGGGTKVEIK |
| | (Vk2) | |

After combining the above variants, each combination was subjected to measurement of a binding ability (that is, affinity). Then, using BIACORE T200 (serial no. 1909913) device, BIACORE T200 Control Software V2.0.1 and BIACORE T200 Evaluation Software V3.0 (GE Healthcare, Uppsala, Sweden) analysis program, affinity of each candidate antibody to the antigen, c-Met, was determined along with a dissociation constant (KD value). After fixing the anti-cMet antibody to be analyzed on a protein ACM5 chip (GE Healthcare, Little Chalfont, UK), sensorgrams were acquired by flowing human c-Met antigen (Sino Biological, Beijing, China) at different concentrations in a range of 0.25 to 2 nM over the antibody. Based on the acquired sensorgrams, rate constants kₒₙ and k_{off} were measured and KD value was calculated from a ratio of k_{off}/kₒₙ. The calculated results are shown in Table 10 below, wherein 10 analyzed antibodies were listed in the order of higher affinity to lower affinity.

TABLE 10

| | | | Relative | | |
|---|---|---|---|---|---|
| | | | KD to | | Rmax |
| | | | VMDO2 | | Chi² |
| No. | Ligand | Kd(M) | VH0/Vk0 | Rmax | (RU²) |
| 1 | VH5/Vk0 | 3.52 × 10⁻¹⁰ | 0.68 | 102.8 | 0.515 |
| 2 | VH4/Vk0 | 3.95 × 10⁻¹⁰ | 0.76 | 70.5 | 0.354 |
| 3 | VH6/Vk0 | 4.14 × 10⁻¹⁰ | 0.8 | 89 | 0.249 |
| 4 | VH5/Vk2 | 4.2 × 10⁻¹⁰ | 0.81 | 65.9 | 0.152 |
| 5 | VH4/Vk2 | 4.23 × 10⁻¹⁰ | 0.81 | 68.1 | 0.177 |
| 6 | VH6/Vk2 | 4.4 × 10⁻¹⁰ | 0.85 | 69.6 | 0.133 |
| 7 | VH2/Vk2 | 5.02 × 10⁻¹⁰ | 0.97 | 73.9 | 0.156 |
| 8 | VH0/Vk2 | 5.12 × 10⁻¹⁰ | 0.98 | 57.1 | 0.115 |
| 9 | VH0/Vk0 | 5.2 × 10⁻¹⁰ | 1 | 64.7 | 0.262 |
| 10 | VH2/Vk0 | 5.34 × 10⁻¹⁰ | 1.03 | 57.5 | 0.128 |

It could be seen that affinity of the candidate antibodies according to the present invention was altered within about 2 times as much, compared to varied antibodies (VH0/Vk0) sharing the same variable region as that of the existing antibody 1E4. Further, it was demonstrated that all of 10 candidate antibodies exhibited high affinity in picomolar level (pM, KD value: 10⁻¹⁰ to 10⁻¹²).

Next, SYPRO® Orange (ThermoFisher, Loughborough, UK) was used to assess thermal stability of the candidate antibodies. Further, a melting point for unfolding the antibody was analyzed by StepOnePlus real-time PCR system (ThermoFisher, Loughborough, UK) while increasing the temperature from 25° C. to 99° C. in 56 minutes. The melting curves were analyzed by Protein Thermal Shift Software version 1.2 (ThermoFisher, Loughborough, UK). The analyzed results are shown in Table 11 below.

As shown in Table 11, it could be seen that stability of the antibody was mostly influenced by the heavy chain region, and the stability was reduced in the order of VH6>VH4>VH5>VH0. Further, with reference to a melting temperature (Tm1) when a fragment antigen binding (Fab) structure of the antibody is unfolded, it could be seen that two candidate antibodies including VH6 as well as the candidate antibodies VH4/Vk0 and VH5/Vk0 exhibit increased thermal stability.

TABLE 11

| | | Average | Average |
|---|---|---|---|
| No. | Antibody | Tₘ1 (° C.) | Tₘ2 (° C.) |
| 1 | VH6/Vk0 | 65.4 | * |
| 2 | VH6/Vk2 | 63.2 | * |
| 3 | VH5/Vk0 | 62.6 | ~68 |
| 4 | VH4/Vk0 | 62.6 | ~68 |
| 5 | VH0/Vk0 | 60 | 67.7 |
| 6 | VH5/Vk2 | 59.7 | 67.3 |
| 7 | VH4/Vk2 | 59.6 | 67.3 |
| 8 | VH0/Vk2 | 58 | 67.5 |

### Example 9—Analysis for Biological Activity (Biopotency) of Candidate Antibodies

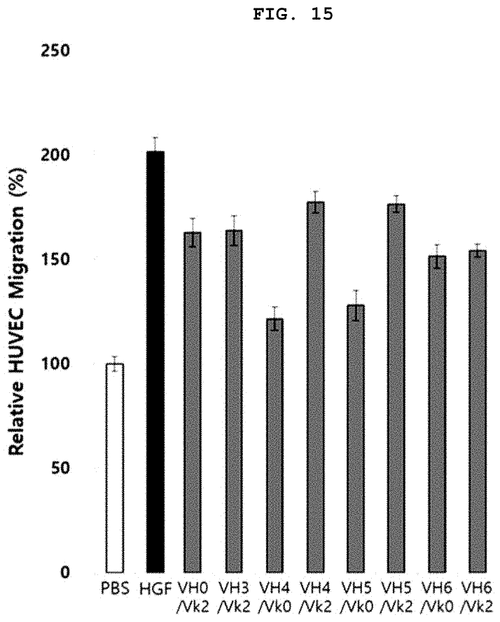

For analogy of biopotency of each candidate antibody, HUVEC migration assay was performed. HUVEC migration assay is a simple examination method constructed on the basis of a principle in which human umbilical vein endothelial cell (HUVEC) migrates when a c-Met protein receptor of a cell is activated by a ligand such as HGF. It may be understood that, as an assay value, that is, % migration is higher, HUVEC migration may be more accelerated. Similar to HGF, the anti-cMet antibody can phosphorylate the antigen, that is, c-Met and activate the same, thereby expecting possibility of inducing cell migration. A transwell (Corning Inc., USA) was coated with 1% gelatin (Sigma Aldrich, USA), followed by spreading HUVEC (Lonza, USA) at 2×10⁴ cells/well thereto. 10% fetal bovine serum (Gibco, New Zealand) was placed in a top chamber of the transwell while Medium 199 added with 1% fetal bovine serum (Gibco, USA) was placed in a bottom chamber of the transwell, followed by incubation at 37° C. under 5% CO₂ condition. After incubation for 50 minutes, the bottom chamber was treated with 50 ng/ml of recombinant human hepatocyte growth factor (rhHGF) (R&D systems, USA) and 90 ng/ml of each candidate antibody, respectively. After incubation under the condition of 37° C. and 5% CO₂ for 2 hours, the cells were fixed with 3.7% formaldehyde (VWR Life Science, USA) and stained with crystal violet (VWR Life Science, USA), followed by observation using a microscope and photographing the same in order to quantify how far HUVEC migrates from an inner side to an outer side of the transwell. The measured results are shown in FIG. 15.

As a result of the experiment, the % migration was about 120 to 179% compared to a negative control group, and therefore, it could be seen that, like the positive control group, that is, recombinant human HGF (rhHGF), the candidate antibodies can accelerate migration of cells (FIG. 15). Accordingly, it can be seen that the candidate antibodies of the present invention have excellent biopotency from the above results.

### Example 10—Data Comparison and Assay for Selection of Final Candidate Antibodies

In order to determine a final candidate group from the above selected candidate antibodies, standards for determination were subdivided into expression level, affinity, thermal stability and biopotency, and used for comparison. Generally, considering production time and costs, the expression level among the above standards is regarded as the most important indicator. Accordingly, in first consideration of smooth antibody production, the present inventors have excluded the candidate antibodies with an expression level of less than 15 ρg/ml. Thereafter, compared to the reference antibody (VH0/Vk0), improvement of each candidate antibody was converted into a numerical value from each of selection indexes. Then, an assay value of the candidate antibody measured from the selection index was divided by an average assay value to obtain a ratio (index 1). This index 1 was again divided by a ratio of VH0/Vk0 to obtain index 2, which in turn, is normalized (Tables 12 to 15). Further, these numerical values analyzed from four selection indexes (that is, the above indicators) were subjected to multiplying operation, and the candidate antibodies were overall compared with respect to the four selection indexes. Therefore, the finally resultant values (index 3) were listed in the order (highest first). As a result, four types of more improved candidate antibodies (VH4/Vk2, VH5/Vk2, VH6/Vk2 and VH6/Vk0) than the existing prototype antibody (VH0/Vk0) were selected (Table 16).

TABLE 12

| | Expression | | |
|---|---|---|---|
| Antibody | level (μg/ml) | Index 1 | Index 2 |
| VH0/Vk0 | 37.1 | 1.213 | 1 |
| VH0/Vk2 | 32.4 | 1.059 | 0.873 |
| VH4/Vk0 | 22.4 | 0.732 | 0.604 |
| VH4/Vk2 | 49.7 | 1.625 | 1.34 |
| VH5/Vk0 | 21.6 | 0.706 | 0.582 |
| VH5/Vk2 | 46.4 | 1.517 | 1.251 |
| VH6/Vk0 | 30.2 | 0.987 | 0.814 |
| VH6/Vk2 | 40.5 | 1.324 | 1.092 |

TABLE 13

| Antibody | KD (10⁻¹⁰ M) | Index 1 | Index 2 |
|---|---|---|---|
| VH0/Vk0 | 5.2 | 0.677 | 1 |
| VH0/Vk2 | 5.12 | 0.688 | 1.016 |
| VH4/Vk0 | 3.95 | 0.891 | 1.316 |
| VH4/Vk2 | 4.23 | 0.832 | 1.229 |
| VH5/Vk0 | 3.52 | 1 | 1.477 |
| VH5/Vk2 | 4.2 | 0.838 | 1.238 |
| VH6/Vk0 | 4.14 | 0.85 | 1.256 |
| VH6/Vk2 | 4.4 | 0.8 | 1.182 |

TABLE 14

| Antibody | Tm1 (° C.) | Index 1 | Index 2 |
|---|---|---|---|
| VH0/Vk0 | 60 | 1.006 | 1 |
| VH0/Vk2 | 58 | 0.973 | 0.967 |
| VH4/Vk0 | 62.6 | 1.05 | 1.043 |
| VH4/Vk2 | 59.6 | 1 | 0.993 |
| VH5/Vk0 | 62.6 | 1.05 | 1.043 |
| VH5/Vk2 | 59.7 | 1.001 | 0.995 |
| VH6/Vk0 | 65.4 | 1.097 | 1.09 |
| VH6/Vk2 | 63.2 | 1.06 | 1.053 |

TABLE 15

| | Relative | | |
|---|---|---|---|
| Antibody | Migration (%) | Index 1 | Index 2 |
| VH0/Vk0 | 149.8 | 0.951 | 1 |
| VH0/Vk2 | 162.8 | 1.034 | 1.087 |
| VH4/Vk0 | 121.7 | 0.773 | 0.812 |
| VH4/Vk2 | 177.4 | 1.127 | 1.185 |
| VH5/Vk0 | 128.1 | 0.813 | 0.855 |
| VH5/Vk2 | 176.5 | 1.121 | 1.179 |
| VH6/Vk0 | 151.5 | 0.962 | 1.012 |
| VH6/Vk2 | 154.2 | 0.979 | 1.03 |

TABLE 16

| Antibody | Index |
|---|---|
| VH4/Vk2 | 1.938 |
| VH5/Vk2 | 1.816 |
| VH6/Vk2 | 1.399 |
| VH6/Vk0 | 1.128 |
| VH0/Vk0 | 1 |
| VH0/Vk2 | 0.932 |
| VH5/Vk0 | 0.767 |
| VH4/Vk0 | 0.674 |

For purpose of understanding technical configurations of the claims, the appended claims described below should not be interpreted narrowly than words applied literally in any way, therefore, illustrative embodiments in the specification could not be directly considered as the claims. Accordingly, it is duly understood that the present invention has been described with reference to illustrative embodiments, which do not limit the scope of the claims. Therefore, the present invention should be only limited by the appended claims. All publications, issued patents, patent applications, books and journal papers cited in the present disclosure are included in the present invention, the entire contents of which are incorporated herein by reference.

A sequence listing electronically submitted with the present application on Dec. 19, 2019 as an ASCII text file named 20191219 Q24219LC46 TU SEQ, created on Dec. 19, 2019 and having a size of 9,000 bytes, is incorporated herein by reference in its entirety.

1: A kit for diagnosing a renal disease or predicting prognosis of the renal disease from urine, comprising:
a binding molecule specifically bound to cMet protein contained in the urine,
wherein, when a level of the cMet protein in the urine is higher than a normal group, this condition is diagnosed as the renal disease or predicted to have poor prognosis of the renal disease by the kit.

2: The kit according to claim 1, wherein the binding molecule is an anti-cMet antibody.

3: The kit according to claim 1, wherein the renal disease is at least one selected from the group consisting of acute kidney injury, end-stage kidney disease (ESKD), systemic erythematous lupus, diabetic nephropathy (DN), IgA nephropathy (IgAN), HIV-relevant nephritis, non-diabetic chronic nephropathy, focal segmental glomerulosclerosis (FSGS), minimal change disease (MCD) and xanthine oxidase deficiency disease.

4: The kit according to claim 1, further comprising a binding molecule specifically bound to creatinine.

5: A composition for diagnosing a renal disease or predicting prognosis of the renal disease from urine, comprising:
a binding molecule specifically bound to cMet protein contained in the urine,
wherein, when a level of the cMet protein is higher than a normal group, this condition is diagnosed as the renal disease or predicted to have poor prognosis of the renal disease by the composition.

6: A method for diagnosing a renal disease or predicting prognosis of the renal disease from urine, comprising:
measuring a level of cMet protein contained in a urine sample obtained from an object,
wherein, when the level of the cMet protein is higher than a normal group, this condition is diagnosed as the renal disease or predicted to have poor prognosis of the renal disease.

7: The method according to claim 6, wherein the measuring of the level of the cMet protein comprises using a binding molecule specifically bound to the cMet protein.

8: The method according to claim 6, wherein the binding molecule is an anti-cMet antibody.

9: The method according to claim 6, wherein the normal group is an object without any renal disease.

10: The method according to claim 6, wherein the renal disease is at least one selected from the group consisting of acute kidney injury, end-stage kidney disease (ESKD), systemic erythematous lupus, diabetic nephropathy (DN), IgA nephropathy (IgAN), HIV-relevant nephritis, non-diabetic chronic nephropathy, focal segmental glomerulosclerosis (FSGS), minimal change disease (MCD) and xanthine oxidase deficiency disease.

11: The method according to claim 6, further comprising measuring a urine creatinine concentration, a total urinary protein concentration, or the urine creatinine concentration and the total urinary protein concentration.

12: A screening method of a substance for treatment of renal disease, comprising:
a first measurement step of measuring a level of cMet protein in urine of an object being a mammal other than a human;
a step of injecting a sample to be analyzed into the object; and
a second measurement step of measuring the level of the cMet protein in urine of the object,
wherein, when the level of the cMet protein in the second measurement step is lower than the level of cMet protein in the first measurement step, the sample to be analyzed is identified as the substance for the treatment of the renal disease.

13: A method for treatment of renal disease, comprising:
measuring a level of cMet protein contained in a urine sample obtained from an object; and
administering an anti-cMet antibody to the object with the level of the cMet protein in the urine sample higher than that of a normal group.

14: The method according to claim 13, wherein the measuring of the level of the cMet protein comprises using a binding molecule specifically bound to cMet.

15: The method according to claim 14, wherein the binding molecule is an anti-cMet antibody.

16: The method according to claim 13, wherein the normal group is an object without any renal disease.

17: The method according to claim 13, wherein the renal disease is at least one selected from the group consisting of acute kidney injury, end-stage kidney disease (ESKD), systemic erythematous lupus, diabetic nephropathy (DN), IgA nephropathy (IgAN), HIV-relevant nephritis, non-diabetic chronic nephropathy, focal segmental glomerulosclerosis (FSGS), minimal change disease (MCD) and xanthine oxidase deficiency disease.

18: The method according to claim 13, further comprising measuring a urine creatinine concentration, a total urinary protein concentration, or the urine creatinine concentration and the total urinary protein concentration.

19: The method according to claim 13, wherein the anti-cMet antibody includes:
a heavy chain complementarity determining region having an amino acid sequence represented by SEQ ID NO: 1;
a heavy chain complementarity determining region having an amino acid sequence represented by SEQ ID NO: 2;
a heavy chain complementarity determining region having an amino acid sequence represented by SEQ ID NO: 3;
a light chain complementarity determining region having an amino acid sequence represented by SEQ ID NO: 4;
a light chain complementarity determining region having an amino acid sequence represented by SEQ ID NO: 5; and
a light chain complementarity determining region having an amino acid sequence represented by SEQ ID NO: 6.

20: The method according to claim 13, wherein the anti-cMet antibody comprises:
a heavy chain variable region having an amino acid sequence represented by SEQ ID NO: 7,9, 10 or 11; and
a light chain variable region having an amino acid sequence represented by SEQ ID NO: 8 or 12.


SEQUENCE LISTING (12 sequences)

SEQ ID NO: 1 - protein, 5 aa, Artificial Sequence
  CDRH1
THWIT

SEQ ID NO: 2 - protein, 16 aa, Artificial Sequence
  CDRH2
TIDPTDSYNFYGPSFQ

SEQ ID NO: 3 - protein, 16 aa, Artificial Sequence
  CDRH3
DGNYYDSRGYYYDTFD

SEQ ID NO: 4 - protein, 11 aa, Artificial Sequence
  CDRL1
RASQGISTYLA

SEQ ID NO: 5 - protein, 7 aa, Artificial Sequence
  CDRL2
SASTLES

SEQ ID NO: 6 - protein, 9 aa, Artificial Sequence
  CDRL3
QQADSFPLT

SEQ ID NO: 7 - protein, 126 aa, Artificial Sequence
  Heavy chain variable region (VH0)
QVQLVQSGAEVKKPGESLRISCQGSGYSFPTHWITWVRQMPGKGLEWMGTIDPTDSYNFYGPSFQGHVTISADSSSSTAYLQWSSLKASDTAMYYCARDGNYYDSRGYYYDTFDMWGQGTLVTVSS

SEQ ID NO: 8 - protein, 107 aa, Artificial Sequence
  Light chain variable region (Vk0)
DIQMTQSPSFLSASVGDRVTITCRASQGISTYLAWYQQKPGTAPKLLIYSASTLESGVPSRFSGSGSGTDFTLTISSLQPEDSATYYCQQADSFPLTFGGGTKVEIK

SEQ ID NO: 9 - protein, 126 aa, Artificial Sequence
  Heavy chain variable region (VH4)
QVQLVQSGAEVKKPGSSVKVSCQGSGYSFPTHWITWVRQAPGQGLEWMGTIDPTDSYNFYGPSFQGRVTITADSSTSTAYMELSSLRSEDTAMYYCARDGNYYDSRGYYYDTFDMWGQGTLVTVSS

SEQ ID NO: 10 - protein, 126 aa, Artificial Sequence
  Heavy chain variable region (VH5)
QVQLVQSGAEVKKPGSSVKVSCQGSGYSFPTHWITWVRQAPGQGLEWMGTIDPTDSYNFYGPSFQGRVTITADSSTSTAYMELSSLRSEDTAVYYCARDGNYYDSRGYYYDTFDMWGQGTLVTVSS

SEQ ID NO: 11 - protein, 126 aa, Artificial Sequence
  Heavy chain variable region (VH6)
QVQLVQSGAEVKKPGSSVKVSCKASGYSFPTHWITWVRQAPGQGLEWMGTIDPTDSYNFYGPSFQGRVTITADKSTSTAYMELSSLRSEDTAVYYCARDGNYYDSRGYYYDTFDMWGQGTLVTVSS

SEQ ID NO: 12 - protein, 107 aa, Artificial Sequence
  Light chain variable region (Vk2)
DIQMTQSPSSLSASVGDRVTITCRASQGISTYLAWYQQKPGKAPKLLIYSASTLESGVPSRFSGSGSGTDFTLTISSLQPEDFATYYCQQADSFPLTFGGGTKVEIK